(19) United States
(12) Patent Application Publication
(10) Pub. No.: US 2016/0361109 A1
(43) Pub. Date: Dec. 15, 2016

(54) METHODS FOR INDUCING ELECTROPORATION AND TISSUE ABLATION

(71) Applicants: Massachusetts Institute of Technology, Cambridge, MA (US); Virginia Tech Intellectual Properties, Inc., Blacksburg, VA (US)

(72) Inventors: James C. Weaver, Sudbury, MA (US); Reuben S. Son, Cambridge, MA (US); Thiruvallur R. Gowrishankar, Acton, MA (US); Daniel C. Sweeney, Blacksburg, VA (US); Rafael V. Davalos, Blacksburg, VA (US)

(21) Appl. No.: 15/179,310

(22) Filed: Jun. 10, 2016

Related U.S. Application Data
(60) Provisional application No. 62/174,118, filed on Jun. 11, 2015.

(51) Int. Cl. A61B 18/14; A61N 1/30

(52) U.S. Cl. CPC A61B 18/1402; A61N 1/303; A61B 2018/00577

(57) ABSTRACT

The invention encompasses a method of inducing a high permeability state in a cell membrane and a method for ablating a target tissue wherein the method comprises applying an electroporation pulse to a cell, wherein at a time after the electroporation pulse is applied, a plurality of long lived pores (LLPs) are formed in the cell membrane and the presence of the LLPs causes a change in the cell osmotic pressure difference. The invention also encompasses a method for ablating a target tissue using an electrical pulse regime that induces cell permeabilization and cell death, wherein the primary mechanism of cell death is as a result of electroporation.

### RELATED APPLICATION

This application claims the benefit of U.S. Provisional Application 62/174,118, filed Jun. 11, 2015. The entire teaching of the above application is incorporated herein by reference.

### GOVERNMENT SUPPORT

This invention was made with Government support under Grant No. R01 GM063857 awarded by the National Institutes of Health. The Government has certain rights in the invention.

### BACKGROUND OF THE INVENTION

Cell membrane nanopores have been demonstrated experimentally using nanosecond electric field pulses¹⁴⁻¹⁷ in addition to conventional electroporation (EP). In spite of this progress, however, electromagnetic field stimulation of cells remains poorly understood. Purely experimental approaches are inefficient and incomplete, because the combined cellular/field parameter space is huge. This motivates the pursuit of multiscale models with increasing complexity and realism. Models offer objective guidance and perspective for investigators, and can provide rapid, relatively inexpensive initial insights into what is important for experimental examination. Models may also yield previews of new or under-appreciated phenomena, and may guide applications.

Electroporation techniques utilize strong electric fields to create pores in the cell membrane and induce an increase in the permeability of the membrane (Jiang et al. (2015), IEEE Transactions on Biomedical Engineering 62(1): 4-20; Son et al. (2016), IEEE Transaction on Biomedical Engineering 63(3): 571-580). Electroporation itself is the phenomenon that occurs in lipid bilayer membranes wherein defects generated through normal thermodynamic membrane fluctuations are created and expanded using the strong, yet brief electric fields (Abidor et al., Bioelectrochemistry Bioenerg 6(1): 37-52, 1979). The expanded defects that are generated during electroporation are referred to as electropores and enable molecular transport to occur across the cell membrane.

Electroporation has been performed in vitro to enhance gene transfection efficiency (Neumann et al., EMBO J, 1(7): 841-845, 1982) and in vivo to directly disrupt cell physiology (Davalos et al., Ann. Biomed. Eng. 33(2): 223-231 2005) to induce cell death or to augment the delivery of chemotherapeutic drugs to a cell within a target tissue (Mir et al., Br J Cancer 77(12): 2336-42, 1998). Irreversible electroporation (IRE) induces irreversible disruption of the cell membrane and results in cell death (Jourbachi et al., Gastrointest. Interv. 3:8-18, 2014). In applying the electric fields to the target tissue, clinicians are typically unable to monitor the permeabilization of cells intra-operatively and rely on pre-treatment modeling (Edd et al., Technol. Cancer Res. Treat. 6(4): 275-86, 2007) and experience from post-treatment analysis (Martin et al., Ann. Surg., 262(3): 486-494, 2015). In order to ensure that cells within the target tissue are adequately permeabilized, clinicians typically apply electric fields beyond what is required to effectively treat the target tissue. This may result in inadvertent thermal damage because when such intense electric fields are applied, excessive electrical current may pass through the resistive tissue causing unwanted heating. When the temperature of the tissue is increased beyond 40° C. for a prolonged period of time, protein denaturation and other thermal damage may occur in physiological cells and tissue (Lebar et al., Electro- and Magnetobiology, 17(2): 255-262, 1998). As such, the electric field parameters used in electroporation-based treatments and therapies, such as irreversible electroporation (IRE), are selected to mitigate this thermal damage by maintaining the tissue temperature below the protein denaturation threshold (Shafiee et al., J Biomech. Eng., vol. 131(7): 074509, 2009).

However, there are challenges associated with the use of IRE for tumor ablation. For example, ablation of large volumes of tissue with IRE remains difficult because the larger electric fields (for example, greater than 2500 V/cm) that would create larger lesions may also damage surrounding nerves and the cardiovascular system (Jiang et al., 2015). In addition, some studies have shown that incomplete treatment can result after IRE, possibly resulting in tumor recurrence (Jiang et al., 2015). Therefore, there remains a need in the art for electroporation methods that can reduce or avoid thermal damage and address some of the limitations of conventional electroporation techniques. In addition, there remains a need for multiscale models, and improved methods of electroporation and nonthermal tissue and tumor ablation.

### SUMMARY OF THE INVENTION

The present invention is based, at least partially, on the discovery that there is a second type of pore involved in electroporation and that a high permeability state can be induced in the cell membrane using the low energy permeabilzation methods described herein. Furthermore, the present inventors have discovered a method for cell disruption using a single electrical pulse that can effectively induce leakage of cytosolic components into the extracellular space following elevated membrane tension and/or post-electroporation swelling of the cell. The methods described herein can, for example, be used to provide an electroporation method that uses reduced electrical energy, and therefore reduces thermal damage generated through Joule heating, as compared to multiple pulse electroporation treatment schemes.

In some embodiments, the invention is directed to a method of inducing a high permeability state in a cell membrane comprising applying an electroporation pulse in a manner that results in a change in the cell osmotic pressure difference. In yet additional embodiments, the method comprises applying an electroporation pulse to a cell, wherein at a time after the electroporation pulse is applied, a plurality of long lived pores (LLPs) are formed in the cell membrane and the presence of the LLPs causes a change in the cell osmotic pressure difference. In certain aspects, after the change in cell osmotic pressure difference, mechanoporation occurs wherein a plurality of the LLPs expand and/or a plurality of new pores are formed, thereby inducing a high permeability state in a region of the outer cell membrane.

In additional embodiments, the invention is directed to a method of ablating a target tissue, such as a tumor, in a subject in need thereof comprising inducing a high permeability state in a target tissue cell membrane, such as a tumor cell membrane, wherein said method comprises applying an electroporation pulse in a manner that results in a change in the cell osmotic pressure difference.

In yet additional embodiments, the invention is a method of performing electrochemotherapy in a subject in need thereof comprising inducing a high permeability state in a cell membrane and administering an effective amount of therapeutic agent, wherein the method comprises applying an electroporation pulse in a manner that results in a change in the cell osmotic pressure difference.

In further embodiments, the invention is a method of applying nanosecond pulsed electric fields in a subject in need thereof comprising inducing a high permeability state in a cell membrane, wherein the method comprises applying an electroporation pulse in a manner that results in a change in the cell osmotic pressure difference.

In yet additional embodiments, the invention is directed to a method of performing irreversible electroporation in a subject in need thereof comprising inducing a high permeability state in a cell membrane, wherein the method comprises applying an electroporation pulse in a manner that results in a change in the cell osmotic pressure difference.

In further embodiments, the invention is directed to a method of performing calcium electroporation in a subject in need thereof comprising inducing a high permeability state in a cell membrane and administering calcium ions, wherein the method comprises applying an electroporation pulse in a manner that results in a change in the cell osmotic pressure difference.

In yet additional aspects, the invention is a method of ablating a target tissue, wherein the method comprises:

a) placing one or more electrodes within or near the target tissue; and

b) applying a single electrical pulse to the target tissue in an amount which is sufficient to induce cell permeabilization and cell death, wherein the primary mechanism of cell death is as a result of electroporation and/or is non-thermal.

In further embodiments, the invention is a method of ablating a target tissue, wherein the method comprises:

a) placing one or more electrodes within or near the target tissue; and

b) applying a plurality of electrical pulses to the target tissue in an amount which is sufficient to induce cell permeabilization and cell death, wherein the primary mechanism of cell death is non-thermal, and/or as a result of electroporation, wherein the plurality of electrical pulses are each applied at least about 0.1 microsecond to at least about one minute apart. In certain aspects, the plurality of electrical pulses is less than eight pulses.

In certain aspects, the invention is directed to a method of ablating a target tissue, wherein the method comprises:

a) placing one or more electrodes within or near the target tissue; and

b) applying ten or fewer electrical pulses to the target tissue in an amount which is sufficient to induce cell permeabilization and cell death, wherein the primary mechanism of cell death is as a result of electroporation and/or is non-thermal. In certain aspects, fewer than eight electrical pulses are applied.

In additional aspects, the invention encompasses a method of ablating a target tissue in a subject in need thereof, comprising the steps of:

a) placing one or more electrodes within or near the target tissue; and

b) applying a single electrical pulse to the target tissue in an amount which is sufficient to induce biphasic cell permeabilization of the cells of the target tissue, wherein cell death is induced, and wherein the biphasic cell permeabilization comprises electroporation and post-electroporation osmotic swelling and leakage of the cells.

In yet another aspect, the invention is directed to a method of ablating a target tissue in a subject in need thereof, comprising the steps of:

a) placing one or more electrodes within or near the target tissue; and

b) applying a plurality of electrical pulses to the target tissue in an amount which is sufficient to induce biphasic cell permeabilization of the cells of the target tissue, wherein cell death is induced and wherein the biphasic cell permeabilization comprises electroporation and post-electroporation osmotic swelling and leakage of the cells, wherein the plurality of electrical pulses are each applied at least about 0.1 microsecond to at least about one minute apart. In certain aspects, the plurality of electrical pulses is less than eight pulses.

In a further aspect, the invention is directed to a method of ablating a target tissue in a subject in need thereof, comprising the steps of:

a) placing one or more electrodes within or near the target tissue; and

b) applying ten or fewer electrical pulses to the target tissue in an amount which is sufficient to induce biphasic cell permeabilization of the cells of the target tissue, wherein cell death is induced and wherein the biphasic cell permeabilization comprises electroporation and post-electroporation osmotic swelling and leakage of the cells. In certain aspects, fewer than eight electrical pulses are applied.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 shows two pore types, TPs and LLPs, with idealized structures.

FIG. 2 shows three poration phases for low energy membrane permeabilization.

FIG. 3 provides a comparison of electroporation techniques.

FIG. 4 describes characteristics of three ranges of time (orders of magnitude) in the pore lifetime.

FIG. 5 summarizes a rationale for the two pore model.

FIG. 6 is a graph showing the mechanical energy landscape.

FIGS. 7 and 8 summarize transitions between TPs and LLPs and three phases of poration.

FIG. 9 shows a 2D cell model of the three phases of poration.

FIG. 10 is a graph showing a preliminary response of the 2D model.

FIG. 11 shows the simulated electric field intensities delivered to cells seeded along a microfluidic chamber with a tapered channel. Cells seeded in this channel (A) experience a linear drop in electric field across the length of this channel (B).

FIG. 12A shows the simulated electric field intensities delivered to cells seeded along a microfluidic chamber with a tapered channel. Cells seeded in this channel (A) experience a linear drop in electric field across the length of this channel.

FIG. 12B shows a quantification of fraction of cells experiencing rupture (Ruptured Fraction %) over time (min). Though treatments were similar, 99 pulses of 10 microseconds each (99×10 microsecond (μs) pulses) and 99 pulses of 100 μs each (99×100 μs pulses) resulted in similar cellular leakage events, though the lower-energy treatments resulted in a delayed rupture. However, the 10 pulses of 10 microsecond each (10×10 μs) and 10 pulses of 100 microseconds each (10×100 μs pulses) generated minimal cell leakage.

FIG. 13 shows the simulated electric field intensities from 0 to 160 V/cm delivered to cells placed between the Pt/Ir electrodes in the Lab-Tek II chamber setup.

FIG. 14 is a graph showing the fluorescence intensity (AU) of nucleic acid-bound PI has a linear correlation with sub-saturation concentrations of PI (μg/ml) in the extracellular medium.

FIG. 15 shows that cells undergo an event several minutes post-treatment with electric fields in which propidium-bound nucleic acids are expelled from the cell. The white arrow indicates the fluorescent material exiting the cell.

FIG. 16 are graphs showing fluorescence intensity (a.u.) over time (s). This figure shows that the application of a single electrical pulse at lower energies than typically used to electroporate cells outright permeabilize cells in a biphasic manner. H4IIE cells exposed to sufficiently intense electrical pulses of a specific duration become permeabilized outright, such as in the cases of 1.0 millisecond (ms) and 0.2 ms pulses delivered at 500 V (1.1 to 1.25 kV/cm) and 1200 V (2.64 to 3 kV/cm), respectively. However, using lower energies causes a biphasic fluorescence pattern to emerge over time, such as in the cases of 1.0 ms and 0.2 ms pulses delivered at 300 V (0.66 to 0.75 kV/cm) and 900 V (1.98 to 2.25 kV/cm), respectively. Black circles indicate the point at which detectable cellular leakage begins.

FIG. 17 shows PI intensification profiles (fluorescence intensity (a.u.) over time (s)) for each of H4IIE cells investigated.

FIG. 18 shows the cell radius distribution of cell sizes of H4IIE cells in suspension.

FIGS. 19A and 19B shows that for similar energy pulses applied along the tapered channel microfluidic device within similar amounts of time (10 s), similar fluorescent intensity profiles are observed. The traces in the large panels indicate the average cellular fluorescent intensity along the length of the channel for 10 pulses of 100 μs pulse widths and 99 pulses of 10 pulse widths delivered in similar amounts of time with amplitudes of 3 kV (0.8 to 2.65 kV/cm) to CHO cells. The traces in the smaller panels indicate the average fluorescence intensity of the cells at a given position within the channel over time and the gray traces are the individual cell traces.

### DETAILED DESCRIPTION OF THE INVENTION

A description of preferred embodiments of the invention follows.

As used herein, the words “a” and “an” are meant to include one or more unless otherwise specified.

Electromagnetic fields interact with military personnel, for example, at the machine/human interface or in combat environments, potentially affecting performance. These include strong interactions at the outer cell membrane that may also couple electrically to intracellular structures, mainly by conduction currents through outer membrane nanopores.¹⁻⁴ The existence of plasma membrane nanopores is supported by numerous experiments with microsecond and longer pulses,⁵ with increasing support for two pore types.⁶⁻¹³

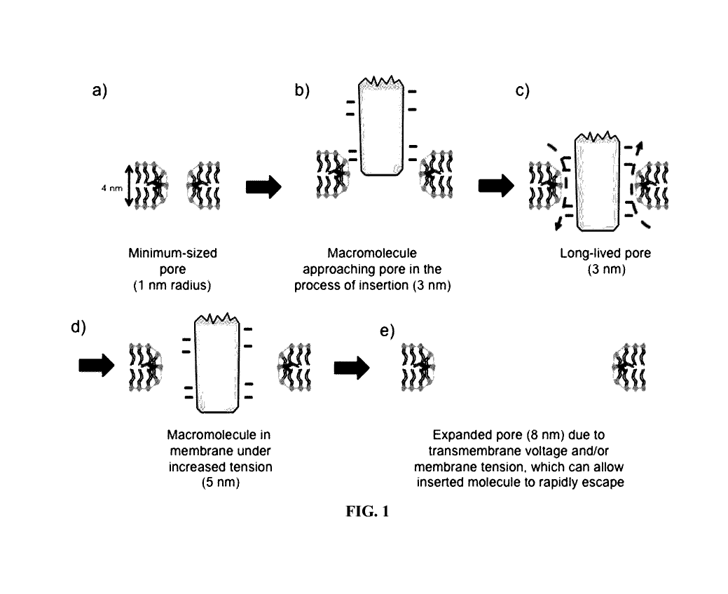

The present invention is based, at least partially, on the appreciation that there is a second type of pore involved in electroporation (EP). The traditional view is that lipidic transient pores (TP) are involved, created in the lipid regions of cell plasma membranes and, for some fields, organelles. There are multiple approaches to cancer treatment using EP including those that modify the immune system and others that aim to non-thermally ablate tumors (resulting in cell death). There is additionally EP work with in vitro cell manipulation. A conceptual model for a second pore type, a long-lived pore (LLP) is shown in FIG. 1.

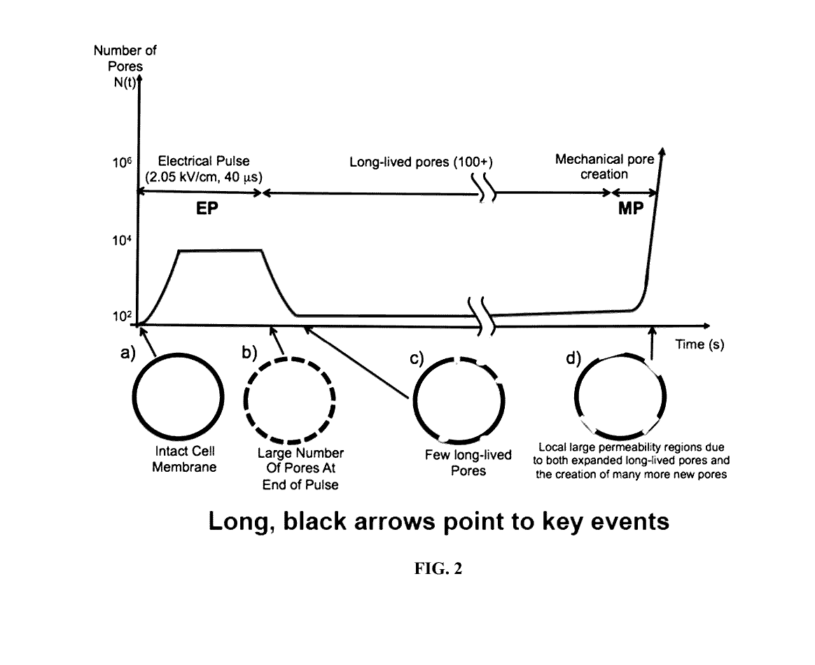

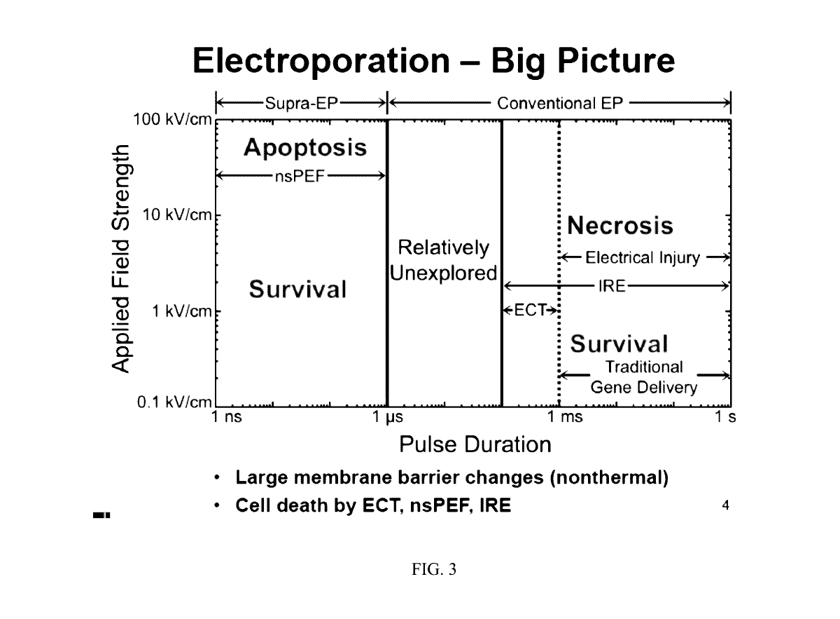

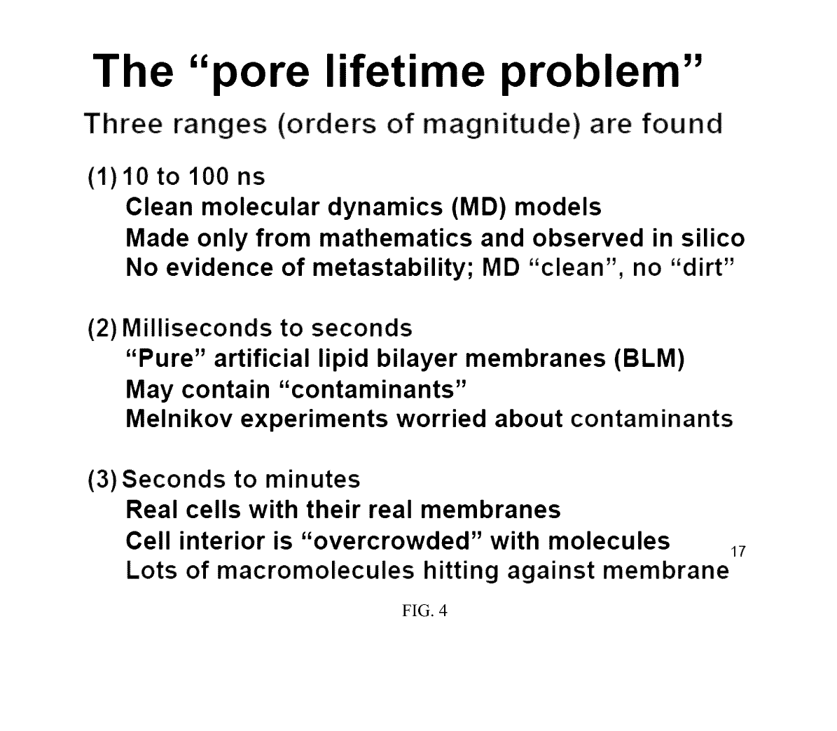

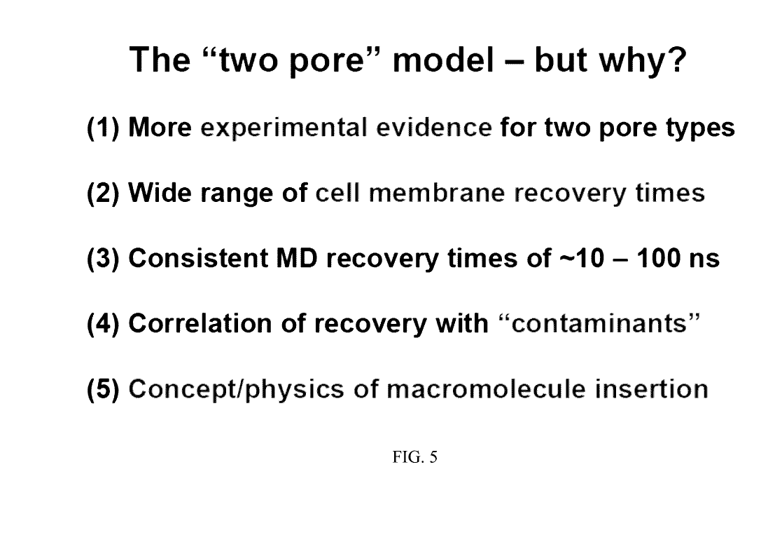

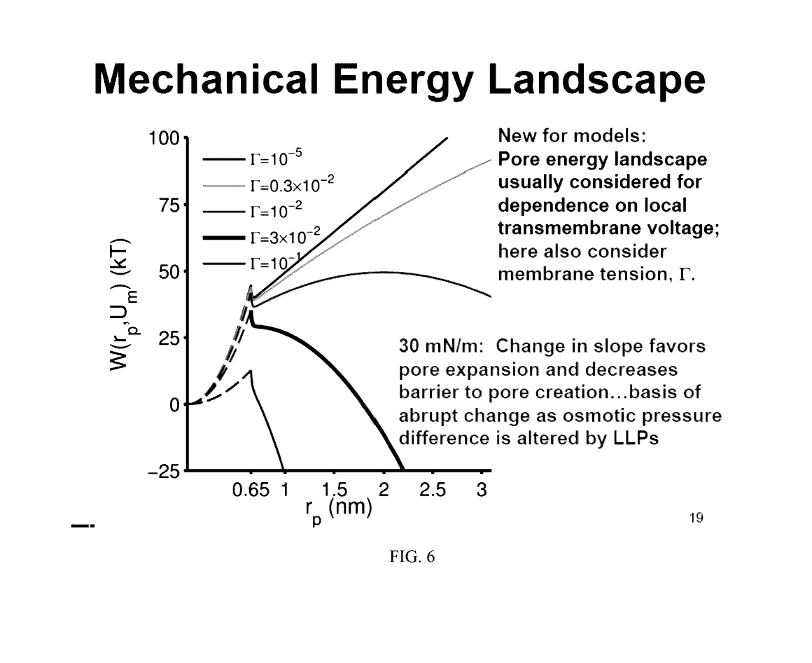

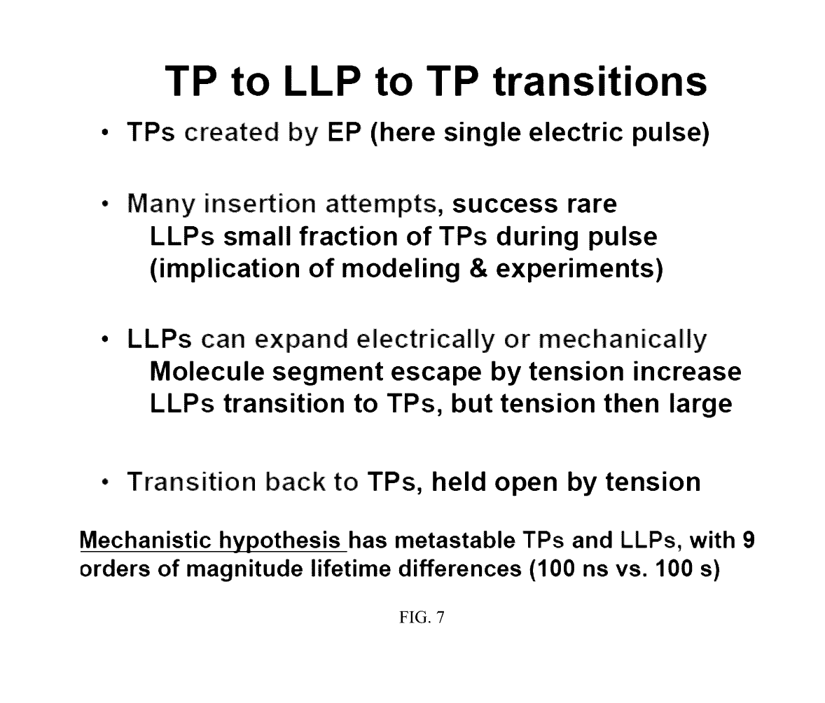

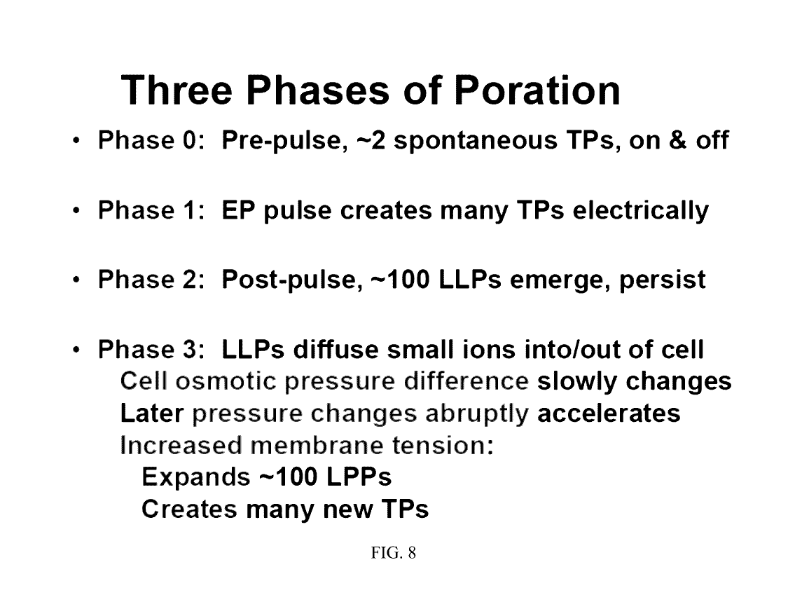

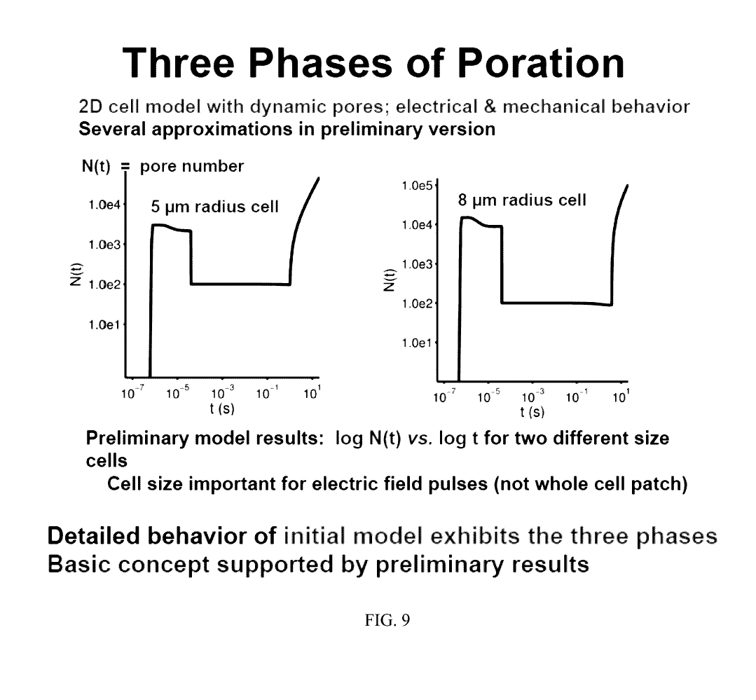

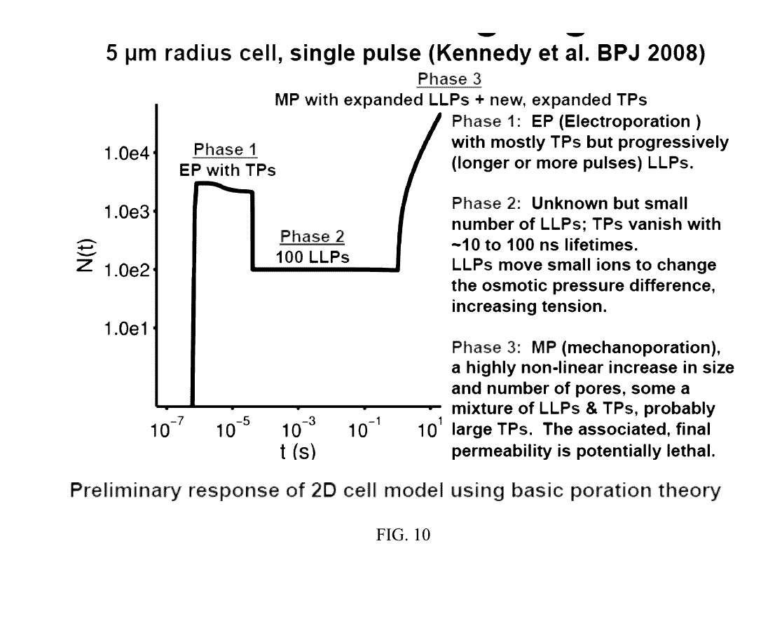

The present invention is also based on the discovery that a large permeability state in a cell membrane can be created after causing a change in the osmotic pressure difference. As discussed in more detail below, this high permeability state involves three phases of poration and involves exploiting intra-/extracellular osmotic pressure differences so that the electrical stimulus (and heating effects) can be smaller, and the change in permeability can be large. A model for the high permeability state is shown in FIG. 2.

A high permeability state in a cell membrane can be induced by a method comprising applying an electroporation pulse in a manner that results in a change in the cell osmotic pressure difference after the electrical pulse is applied. The change in the cell osmotic pressure difference can be such that mechanoporation occurs. As used herein, an electroporation pulse is an electrical pulse that induces electroporation. In certain embodiments, the method comprises applying an electroporation pulse to a cell, wherein at a time during or after the electroporation pulse is applied, a plurality of long lived pores (LLPs) are formed in the cell membrane and the presence of the LLPs causes a change in the cell osmotic pressure difference. In certain aspects, after the change in cell osmotic pressure difference, mechanoporation occurs wherein a plurality of the LLPs expand and/or a plurality of new pores are formed, thereby inducing a high permeability state in one or more regions of the outer cell membrane. In some embodiments, the electroporation pulse is applied for 40 microseconds at 2.05 kV/cm. In yet additional embodiments, cell death occurs after the induction of the high permeability state.

In yet additional embodiments, the invention is directed to a device for inducing a high permeability state. In some embodiments, the invention is directed to a device for inducing a high permeability state in a cell membrane comprising a set of electrodes and a voltage generator, wherein the device induces the high permeability state.

The low energy permeabilization method (high permeability state) described herein can be used for the ablation of a target tissue, for example, for tumor ablation. For example, low energy permeabilization can be used to improve electrochemotherapy (ECT), nanosecond pulsed electric fields (nsPEF), irreversible electroporation (IRE), and/or calcium electroporation. Electrochemotherapy allows the delivery of nonpermeant drugs into a cell and involves the application of short and intense electrical pulses that transiently permeabilize tissue cells (Mir et al., 1999. Mechanisms of Electrochemotherapy, Adv. Drug Deliv. Rev. 35(1): 107-118; the contents of which are expressly incorporated by reference herein). Drugs that can be administered using electrochemotherapy are nonpermeant, cytotoxic drugs (Id.; Sersa et al., 2003, Electrochemotherapy: advantages and drawbacks in treatment of cancer patients, Cancer Therapy 1: 133-142; the contents of which are expressly incorporated by reference herein). Examples of drugs that can be delivered using electrochemotherapy are bleomycin and cisplatin (Mir et al.). Nanosecond pulsed electric fields (nsPEF) utilize short pulses of low energy electric fields (Nuccitelli et al., 2006, Nanosecond pulsed electric fields cause melanomas to self-destruct, Biochem Biophys Res Commun 343(2): 351-360; the contents of which are expressly incorporated by reference herein). nsPEF is often characterized by little heat production and allowing the targeting of intracellular organelles which can lead to apoptosis (Id.). Calcium electroporation is electroporation with calcium and can cause ATP depletion and cell death (Hansen et al., 2015, Dose-Dependent ATP Depletion and Cancer Cell Death Following Calcium Electroporation, Relative Effect of Calcium Concentration and Electric Field strength, PLoS One 10(4): e0122973). Irreversible electroporation (IRE) involves subjecting a cell to an electrical field using high-voltage direct current creating multiple holes in the cell membrane and causing cell death (Narayan, 2011, Irreversible Electroporation for Treatment of Liver Cancer, Gastroenterol. Hepatatol., 7(5): 313-316). NanoKnife® system (Angiodynamics) utilizes IRE. These tumor ablation methods can thus be modified by changing the electrical pulsing protocol to use smaller and/or fewer pulses (resulting in less tissue heating and less nerve stimulation) using the methods described herein, for example, such that a change in osmotic pressure difference results.

With respect to IRE, while enjoying a recent, rapid introduction into the clinic, IRE (irreversible electroporation)^{1B, 2B} has been criticized by some, claiming two potentially serious problems. A major attraction of IRE is that essential structures such as major blood vessels can be spared. However, a question remains as to whether essential structures are spared if there is significant heating (e.g. Temperature (T)>42° C.) for a relatively long time. While many publications conclude that IRE is safe, the description of IRE as “non-thermal” is now explicitly challenged.^{5B,6B} The basic notion is that above ˜42° C. accumulation of damage can occur, such as denaturation of proteins.^{7B} A recent paper reports of a large number/percentage of tumor recurrence, and presents a model which shows that electrically significant major blood vessels perturb the tissue-level fields, due to the high electrical conductivity of blood. This means that some regions near these blood vessels do not experience fields that kill nearby cancer cells.^{8B} This problem appears analogous to thermally significant blood vessels in hyperthermia, wherein the cooling by blood flow in such vessels prevents complete cell killing.^{9B} Complicating matters, EP-induced vascular lock^{10B} should also be relevant, as the bioheat equation for effective heat transfer collapses into the less effective passive diffusion heat transfer if perfusion is stopped for a clinically-relevant time.

In enhanced IRE, the use of electrical pulses that simultaneously create a small temperature rise and a much more homogeneous electric field in tissue at the sites of cells as compared to that with conventional EP used in IRE while preserving the feature of cell death by “accidental necrosis” rather than programmed cell death subroutines.^{3B} A pulsing protocol can be engineered (approximately) by predicting fields near and within cell models, and also by considering non-thermal cell death mechanisms.^{3B} For example, one can purposefully aim to avoid creating and relying on apoptotic cells that lead to the complete process of apoptosis (apparently involving two types of intrinsic apoptosis^{4B}), because macrophages would then be needed to show up in significant numbers to complete the job. Instead, nonthermal accidental necrosis due to nsPEF (nanosecond pulsed electric fields) can be useful, assuming that skin tumors and avoiding scarring are not dominant issues.

The invention encompasses methods for ablating a target tissue, for example, a tumor. The methods can comprise inducing a high permeability state in the cell membrane of the cells of the target tissue. For example, the high permeability state can be induced by applying an electroporation pulse in a manner that results in a change in the cell osmotic pressure difference as described herein. In certain aspects, the electrical pulse(s) used to induce the high permeability state are lower energy pulses (for example, shorter duration and/or lower amplitude) than those used in conventional electroporation methods, for example, those currently used in irreversible electroporation. In yet additional aspects, the method comprises applying a single electrical pulse, ten or fewer electrical pulses, or a plurality of electrical pulses applied at least about 0.1 microsecond to at least about one minute apart, as described herein.

In yet additional aspects, ablation of a target tissue can comprise applying a single electrical pulse, ten or fewer electrical pulses, or a plurality of electrical pulses, as described herein, such that cell death is induced, wherein the primary mechanism of cell death is non-thermal, and/or as a result of electroporation. As described herein, because the methods utilize lower electrical energy than other ablation methods, thermal damage to the tissue can be mitigated. The primary mechanism of cell death is non-thermal when the mechanism of cell death for the majority of the cells in the target tissue is non-thermal. The primary mechanism of cell death is by electroporation when the mechanism of cell death for the majority of the cells in the target tissue is due to electroporation (for example, as opposed to thermal effects).

Irreversible electroporation (IRE) has been described extensively in the literature (see, for example, U.S. Pat. Nos. 8,048,067, 8,282,631, 8,926,606, and 9,005,189; the contents of each of which are expressly incorporated by reference herein). Conventional electroporation techniques for tissue destruction involve multiple pulse regimes and most electroporation studies have used an electric field between 1000 and 2500 V/cm, a pulse duration from 50 to 100 pec and pulse numbers between 10 and 90 (Jiang et al. (2015), IEEE Transaction on Biomedical Engineering 62(1): 3-20; the contents of which are expressly incorporated by reference herein). It has surprisingly been found that the application of a single, low energy electrical pulse can induce biphasic cell permeabilization comprising electroporation, and leakage of cytosolic components into the extracellular space following an expansion of the cell volume or an elevation in membrane tension. The expansion of the cell volume that occurs after electroporation is also referred to herein as post-electroporation osmotic swelling. The leakage of cellular components in the extracellular space that is preceded by the expansion in cell volume can be referred to herein as “leakage of the cells” or “leakage.” The leakage event depends both on the pulse width (duration of the pulse) and amplitude of the applied electric field. As described in more detail below, lower energy treatment results in a biphasic response comprising delayed rupture as compared with higher energy electric fields. This delayed rupture can occur several minutes post-treatment after destabilization of the membrane. Conventional electroporation (for example, multiple pulse, higher amplitude and/or longer duration) regimes can result in monophasic cell permeabilization wherein the cell leakage event occurs continuously post-treatment and reaches an asymptote value over time. In contrast, certain electric field intensities, including specific electric pulse durations and amplitudes, result in biphasic permeabilization comprising an initial electroporation event followed by osmotic swelling and leakage events that can occur several minutes post-treatment.

When the one or more electrodes are placed “near” the target tissue, the electrodes can be placed sufficiently close to the target tissue such that application of an electrical pulse can cause target tissue cell death and/or induce electroporation of the cells of the target tissue. An electrical pulse is applied in “an amount which is sufficient” to achieve or result in a recited effect (for example, to induce biphasic cell permeabilization and/or to induce cell death) when the pulse parameters and/or pulse strength (for example, the number, amplitude and/or duration of the pulse(s)) is sufficient to induce the recited effect. Where the method is described as comprising the application of a single electrical pulse, only one electrical pulse is applied to the target tissue during the same electroporation treatment session (for example, the same IRE treatment session), the same electroporation ablation session (for example, the same IRE ablation session), and/or during the total electroporation treatment time (for example, the total IRE treatment time). Where the method is described as comprising application of a specific number of pulses, for example, two pulses, no additional electrical pulses are applied to the target tissue during the same electroporation treatment session (for example, the same IRE treatment session), the same electroporation ablation session (for example, the same IRE ablation session), and/or during the total electroporation treatment time (for example, the total IRE treatment time).

The invention encompasses methods of ablating a target tissue in a subject in need thereof, comprising the steps of a) placing one or more electrodes within or near the target tissue; and b) applying a single electrical pulse to the target tissue in an amount which is sufficient to induce biphasic cell permeabilization of the cells of the target tissue, wherein cell death is induced, and wherein the biphasic cell permeabilization comprises electroporation and post-electroporation osmotic swelling and leakage of the cells. In some cases, an electrical pulse is applied in an amount that has been predetermined to be sufficient to induce biphasic cell permeabilization. In some aspects, biphasic cell permeabilization of the cells of the target tissue is induced when the majority of the cells (greater than half of the cells) have a biphasic response.

The invention also encompasses a method of ablating a target tissue in a subject in need thereof, comprising the steps of: a) placing one or more electrodes within or near the target tissue; and b) applying a plurality of electrical pulses to the target tissue in an amount which is sufficient to induce biphasic cell permeabilization of the cells of the target tissue, wherein cell death is induced and wherein the biphasic cell permeabilization comprises electroporation and post-electroporation osmotic swelling and leakage of the cells, wherein the plurality of electrical pulses are each applied at least about 0.1 microsecond to at least about one minute apart. In additional aspects, the plurality of electrical pulses are applied at least about 1 microsecond to at least about one minute apart, at least about 10 microseconds to at least about one minute apart, or at least about 100 microseconds to at least about one minute apart. In yet additional aspects, the plurality of electrical pulses are each applied at least about 10 seconds, at least about 20 seconds, at least about 30 seconds, at least about 45 seconds, or at least about one minute apart. In certain aspects, the plurality of electrical pulses are each applied at least about one minute apart. In some cases, the electrical pulses that are applied are sufficient to or have been predetermined to be sufficient to induce biphasic cell permeabilization. In some aspects, the plurality of electrical pulses is less than about 30 pulses, less than about 25 pulses, less than about 20 pulses, less than about 15 pulses, or less than about 10 pulses. The number of pulses can also be less than nine pulses, less than eight pulses, less than seven pulses, less than six pulses, less than five pulses, less than four pulses, or less than three pulses. The number of pulses applied can also be two pulses. When the plurality of pulses are described as being applied a specific time apart, for example, about 0.1 microsecond to at least about one minute apart, the plurality of pulses are each applied with separations of the specific recited time(s), for example, separations of 0.1 microsecond to about one minute.

In yet additional embodiments, the invention is directed to a method of ablating a target tissue in a subject in need thereof, comprising the steps of: a) placing one or more electrodes within or near the target tissue; and b) applying ten or fewer electrical pulses to the target tissue in an amount which is sufficient to induce biphasic cell permeabilization of the cells of the target tissue, wherein cell death is induced and wherein the biphasic cell permeabilization comprises electroporation and post-electroporation osmotic swelling and leakage of the cells. The number of pulses can also be less than ten pulses, less than nine pulses, less than eight pulses, less than seven pulses, less than six pulses, less than five pulses, less than four pulses, or less than three pulses. The number of pulses applied can also be two pulses.

As described above, the method of the present invention can utilize less electrical energy than conventional electroporation protocols, for example, conventional IRE pulse protocols. In some cases, the amplitude or electric field strength of the single electrical pulse or each of the electrical pulses applied according to the present invention can be less than that of an IRE pulse protocol that induces monophasic cell permeabilization for the same target tissue under the same circumstances. For example, the amplitude or the electric field strength of the single or each of the pulses can be less than about 2%, less than about 5%, less than about 7%, less than about 10%, less than about 15%, less than about 20%, less than about 25%, less than about 30%, less than about 35%, less than about 40%, less than about 45% or less than about 50% of the amplitude or electric field strength for an IRE pulse protocol that induces monophasic cell permeabilization for the same target tissue under the same circumstances. In addition, or alternatively, the duration of the single electrical pulse or each of the electrical pulses applied can be less than that of an IRE pulse protocol that induces monophasic cell permeabilization for the same target tissue under the same circumstances. For example, the duration of the single or each of the pulses can be less than about 2%, less than about 5%, less that about 10%, less than about 15% or less than about 20% of the pulse duration for an IRE pulse protocol that induces monophasic cell permeabilization for the same target tissue under the same circumstances. It is known in the art that the effect of an electrical pulse depends on several factors including field amplitude, polarity, number of pulses, shape of the pulses, pulse duration or length, pulse intervals, environmental temperature, cell type, morphology, age and size (Goldberg et al., Biomedical Engineering Online 9:13, pp 1-13, 2010). Mathematical models have been described in the literature that calculate the electrical potential distribution in tissue during typical electroporation pulses (the Laplace equation) and a modified Pennes (bioheat) equation to calculate the resulting temperature distribution (see, for example, U.S. Pat. No. 8,046,067). Thermal damage can also be calculated using Equations 9 and 10 described in U.S. Pat. No. 8,046,067:

Ω=∫ξe^{-Ea/RT}dt (9)

Ω=tₚξe^{-ΔE/RT} (10);

where Ω is a measure of thermal damage, is the frequency factor, Eₐ is the activation energy and R is the universal gas constant. A detailed description on the various degrees of thermal damage as described in Equation (9) (also referred to as an Arrhenius type equation) above can be found in (Diller, K. R., Modeling of bioheat transfer processes at high and low temperatures, in Bioengineering heat transfer, Y. I. Choi, Editor. 1992, Academic Press, Inc: Boston. p. 157-357). Treatment planning has been described as essential for IRE (Jourabachi et al., Gastrointest. Interv. 3:8-18, 2014). Planning an IRE pulse protocol can involve mathematical formulae, such as those based on a deterministic model, using a deterministic single value for the amplitude of the electric field that would be required in order to cause cell death (Jourabachi et al.). Goldberg et al. (Biomedical Engineering Online 9:13, pp 1-13, 2010) proposed a methodology for evaluating cell death in a volume of tissue treated by IRE using a statistical cell death model (Goldberg et al.). U.S. Pat. No. 8,048,067 describes mathematical models and experiments used to determine the maximal extent of tissue ablation that can be accomplished by IRE before thermal effects occur. In certain aspects, the IRE pulse protocol that induces monophasic cell permeabilization is determined based on a modified Pennes bioheat equation and an Arrhenius bioheat equation.

The single electrical pulse or electrical pulses can be applied using one or more electrodes. Where one electrode is used, a reference electrode can also be used. A voltage generator can be used to apply a voltage which provides an electric field around the target tissue in a manner sufficient to induce cell death. The electrodes can be plate, needle, clamp or catheter electrodes. The electrode can be a bipolar (single) electrode or monopolar (single) electrode applicators wherein two electrodes constitute a monopolar electrode pair. Where monopolar electrodes are used, the number of electrodes used can be two (in other words, an electrode pair) or greater. The methods described herein can comprise placing a first electrode and a second electrode within or near the target tissue such that the target tissue is positioned between the first and second electrodes. Where more than two electrodes are used, the electrodes can be placed within or near the target tissue such that the target tissue is positioned between the electrodes. In some aspects, two electrodes, four electrodes, six electrodes, or eight electrodes can be used. The electrodes can be different shapes and sizes and be positioned at various distances from each other. The distance of one electrode from another can be about 0.5 to about 10 cm, about 1 to about 5 cm, or about 2 to about 3 cm. The electrodes can be different distances from each other. The shape of the electrodes can, for example, be circular, oval, square, rectangular or irregular. The size, shape and distances of the electrodes can affect the voltage and pulse duration that should be used and, as such, the pulse parameters can be adjusted accordingly. Wherein at least two electrodes are used, the first electrode can be placed at about 4 mm to 10 cm from the second electrode. In addition, the one or more electrodes can be placed within or near the target tissue under computed tomography (CT) guidance or ultrasound guidance. The electrode (and reference electrode), or electrodes can be part of a single device. An exemplary device is the NanoKnife® system (AngioDynamic, Queensbury, N.Y.) which includes an IRE generator and up to six electrode probes. The Nanoknife system transmits direct current energy from the generator to electrode probes placed in the target area.

As discussed above, the methods described herein can result in thermal damage to the target tissue and/or the surrounding tissue and structures. In some aspects, the methods described herein result in less thermal damage than that induced by an IRE pulse protocol that induces monophasic permeabilization. The decreased thermal damage is, at least partially, due to the shorter pulse duration, pulse length, lower amplitude, lower electrical field strength, decreased number of pulses, lower pulse frequency to allow for heat dissipation, and/or lower total energized time during the procedure. In certain aspects, the single electrical pulses or the electrical pulses are applied in an amount which maintains the temperature of the target tissue at about 65° C. or less. In additional aspects, the single electrical pulse or electrical pulses are applied in an amount which maintains the temperature of the target tissue at about at about 50° C. or less. In yet additional aspects, the single electrical pulse or the electrical pulses are applied in an amount which maintains the temperature of the target tissue at about 45° C. or less, or about 42° C. or less, or about 40° C. or less.

The pulse duration for the single pulse or each of the pulses can be between about 1 nanosecond and about 1 second. In certain aspects, the duration of the single electrical pulse or each of the electrical pulses can be between about 1 microsecond to about 70 milliseconds, between about 5 microseconds to about 70 milliseconds, or between about 10 microseconds to about 70 milliseconds. The duration of the single electrical pulse or each of the electrical pulses can be between about 1 microsecond to about 10 milliseconds, between about 10 microseconds to about 10 milliseconds, between about 20 microseconds to about 10 milliseconds, between about 100 microseconds to about 20 milliseconds, between about 100 microseconds to about 5 milliseconds, between about 20 to about 200 microseconds, between about 50 to about 150 microseconds, or between about 50 to about 100 microseconds. The pulse duration of each of the multiple or plurality of pulses can be the same or different. In certain aspects, the pulse duration of each of the multiple or plurality of pulses is the same.

Exemplary electric field strengths of the single electrical pulse or each of the electrical pulses used according the present invention are between about 100 to about 5000 V/cm. In some aspects, the electric field strength is between about 200 to about 3000 V/cm. The electric field strength can also, for example, be between about 400 V/cm to about 10,000 V/cm, about 400 V/cm to about 3000 V/cm or about 400 V/cm to about 1000 V/cm. The field strength of each of the multiple or plurality of pulses can be the same or different. In certain aspects, the field strength of each of the multiple or plurality of pulses is the same.

The current can, for example, be between about 2 to about 100 A. In certain aspects, the current is between about 2 to about 50 A, or about 50 to about 100 A.

It is to be understood that when the range or amount of a parameter, such as pulse duration, amplitude, electric field strength and current, is described as “between” or “from” a low end of the range to a high end of the range, the range is meant to be inclusive of both the low end and the high end as well as those values in between the low and high ends. For example, when pulse duration is described as between about 20 microseconds to about 10 milliseconds, the range includes both about 20 microseconds and about 10 milliseconds as well as the times in between.

The methods described herein can be used for the ablation of a target tissue. The subject being treated can be a human subject (also referred to herein as a patient) or a veterinary subject. The human subject can be a pediatric patient or an elderly patient. A pediatric patient can be a patient that is 18 years old or younger, or 15 years old or younger, or 12 years old or younger. The elderly patient can be a patient that is 65 years old or older. The target tissue can be a non-malignant or malignant. In some aspects, the target tissue is a tumor or a part of a tumor, including, but not limited to, a soft tissue tumor or a part thereof. Exemplary tumors include tumors of the lung, tumors of the liver, tumors of the kidney, tumors of the pancreas, prostate tumors, breast tumors, colorectal tumors, peri-biliary tumors, melanoma, head and neck and thyroid tumors. In certain aspects, the subject is suffering from breast cancer, colorectal liver metastasis, head and neck cancers, hepatocellular carcinoma, pancreatic cancer, bone cancer, lung cancer, soft tissue cancer, melanoma, peri-biliary tumor, prostate cancer, renal cell carcinoma, renal mass and uveal melanoma. In yet additional aspects, the subject is suffering from locally advanced pancreatic cancer. In further aspects, the tumor is a liver tumor located less than about 1 cm from a major bile duct. The methods described herein can allow the treatment of larger tumors (greater tumor volumes) than that which can be treated by an IRE pulse protocol that induces monophasic cell permeabilization because the risk and extent of thermal damage is less when the electroporation methods described herein are utilized. In certain embodiments, the volume of the target tissue can be about 10 cm³ or greater, about 15 cm³ or greater, about 30 cm³ or greater, or about 50 cm³ or greater. In yet additional aspects, the diameter of the target tissue is about 3 cm or greater. In certain additional embodiments, the volume of the target tumor can be about 10 cm³ or greater, about 15 cm³ or greater, about 30 cm³ or greater, or about 50 cm³ or greater. In yet additional aspects, the diameter of the target tumor is about 3 cm or greater.

In certain additional aspects, the target tissue is cardiac tissue. The method can, for example, be used for ablation of vascular smooth muscle (VSMC). In certain additional aspects, the methods described herein can be used to treat benign prostatic hyperplasia (BPH). In yet additional aspects, the target tissue is adipose tissue. In further aspects, the method is used to reduce subcutaneous fat deposits.

Muscular contractions of the treated subject can also be reduced by using the electroporation methods of the present invention as compared with those that occur using an IRE pulse protocol that induces monophasic cell permeabilization. In some cases, a neuromuscular blocking agent is not administered to the subject.

The ablation procedure can be monitored during and/or after treatment using magnetic resonance imagery (MRI), ultrasound, and/or CT. Such monitoring can be used during electrode placement, to monitor the extent of ablation, and/or to detect untreated residual tumor.

An adjuvant can be administered to the subject before, during or after the application of the electrical pulse(s) of the present invention. The adjuvant can, for example, be a chemotherapeutic drug. Exemplary chemotherapeutic drugs are bleomycin, neocarcinostatin, suramin, and cisplatin. The chemotherapeutic drug can, for example, be administered by parenteral injection or oral administration. The adjuvant can also be an agent that directly modifies membrane properties (for example, line tension and surface tension) such as, surfactants; and agents that impede the resealing process (large molecules, channel holders and the like). Surfactants include, for example, DMSO, polyoxyethylene glycol (C₁₂E₈), and sodium dodecyl sulfate (SDS). An agent that has a channel effect includes gramicidin D. Agents that are pore holders include a-hemolysin, heparin, and sodium thiosulfate. In certain additional aspects, the adjuvant can also be calcium ions, or a solution comprising calcium ions. In certain aspects, the adjuvant can be an agent that causes osmotic swelling. An exemplary agent that causes osmotic swelling is deionized (DI) water.

It is to be understood that specific embodiments described herein can be taken in combination with other specific embodiments delineated herein.

The invention is illustrated by the following examples which are not meant to be limiting in any way.

### EXEMPLIFICATION

### Example 1

### Long Lived Pores (LLP) and the High Permeability State

The high permeability state involves three phases of poration and involves exploiting intra-/extracellular osmotic pressure differences so that the electrical stimulus (and heating effects) can be smaller, and the change in permeability can be large. A model for the high permeability state is shown in FIG. 2. LLPs are involved as they allow EP to trigger mechanoporation (MP). The conceptual model is supported by quantitative simulations using an approximate cell model that includes dynamic EP with both TPs (traditional transient pores) and the LLPs. The initial simulations support the complex sequence of:

Phase 1: 40 microsecond EP pulse

Phase 2: Intervening time in which most TPs vanish, and about 100 LLPs survive. These LLPs supply/remove Na+, K+ and Cl− ions, causing a change in the cell osmotic pressure difference.

Phase 3: After some time, there is a nonlinear acceleration in LLP expansion, and then new TP creation, with the combination leading to high permeability states in some local regions of an outer cell membrane.

Cell level continuum modeling predict electrical, poration and solute transport behavior at one or more cell membranes, with simple or irregular membrane geometry.¹⁸⁻²⁴ These are performed for isolated cells with an outer (plasma) membrane, one or more organelle membranes, and multiple membranes of cells close together (e.g in vivo conditions).²⁴ Present capability includes predictions of measurable quantities (transmembrane voltage, Δφm, membrane conductance, Gₘ, and cumulative solute transport, nₛ), and also internal quantities not accessible to measurement (e.g. nanopore size distributions). This and the extensions outlined below can be used with import of MD (molecular dynamics) functional results that are appropriately extrapolated for different nanopore sizes (radii of ˜1 to ˜60 nm), and a wide range of times (˜1 ns to ˜1,000 s).

FIG. 1 shows a conceptual model supported by quantitative simulations. It is consistent with growing evidence for two types of nanopores (“pores” for brevity) in electroporation (EP). In established models, there are only transient pores (TPs; FIG. 1a); here we add explicit long-lived pores (LLPs; FIG. 1c).⁶⁻¹³ The second is developing a unifying hypothesis for cell poration. It is based on TPs and LLPs for EP, and after EP, delayed mechanoporation (MP) due to increased membrane tension.²⁵⁻²⁷ This identifies a complex sequence for cell permeabilization (FIG. 2). Due to EP, a cell's osmotic pressure difference grows, leading to mechanoporation (MP),²⁵⁻²⁷ and large local permeabilities.

Simple geometries (FIG. 1) convey concepts and underlie approximate continuum models, but for realism MD (molecular dynamics) simulations are also needed. For LLP creation, MD could use a tethered (one atom assigned a huge mass) macromolecule segment near an MD membrane patch, with electrical conditions likely to create a TP^{14, 28-35} (FIG. 1a), so that insertion of a charged molecule tip (segment) is likely, converting a TP into a LLP (FIG. 1b). If this configuration can be stabilized,³⁴ transport of Na⁺, K⁺ and Cl⁺ through the fluctuating gap (FIG. 1c) can be examined. Insertion should be aided by a TP's focusing field due to the spreading/access resistance,³⁶⁻⁴¹ expected at a nanopore for large transmembrane voltages (˜0.5-2 V) during an EP pulse.^{23, 42-44}

Partially occluded TPs have been suggested qualitatively,^{6,7} with quantitative support from Born energy estimates⁴⁵ that extend Parsegian's analysis,^{46,47} and from skin EP experiments that introduced macromolecules to alter and prolong small charged molecule transport through the multilamellar lipids of the stratum corneum.^{48, 49} Here a LLP is created by temporary insertion of a macromolecule segment of a cytoplasmic or extracellular macromolecule during an EP pulse (FIGS. 1a,b,c). Small ions and molecules move through a fluctuating gap (FIG. 1c; red dashed, curved arrows). The gap (FIG. 1d) should depend on transmembrane voltage, membrane tension, charge distribution, macromolecule size/geometry and chemical composition. For large gaps the segment should escape (FIG. 1e), yielding LLP destruction by reversion to a TP. During each EP pulse, only a small fraction of TPs are converted to LLPs (FIG. 1b), so that additional pulses create more LLPs, consistent with recent experiments.¹¹ Many macromolecules are present in large numbers within the over-crowded cytoplasm, continuously jostling and striking the inner leaflet of the cell plasma membrane,⁵⁰ a basis for a large attempt rate. This is also the likely basis for electro-insertion of some macromolecules permanently into cell membranes⁵¹⁻⁵⁴ (FIG. 1c with negligible gap).

FIG. 2 shows poration phases for low energy membrane permeabilization. Motivating EP experiments,^{17,55} report delayed, additional permeabilization. FIG. 2c shows TPs vanishing quickly (˜100 ns) post-pulse, consistent with MD simulations. The few LLPs bridge two poration events, EP and MP. The small ions Na⁺, K⁺ and Cl⁻ move through LLPs by electrodiffusion to change the intra-extracellular osmotic pressure difference. Presently we omit membrane reserves, which act to delay/prevent the increase in membrane tension.⁵⁶ With this omission, small ion diffusion through LLPs leads to increased membrane tension that rapidly reaches “lytic values”,²⁵⁻²⁷ with an abrupt transition to pore expansion and pore creation that creates local high permeability states (FIG. 2d). This occurs by redirecting physio-chemical (osmotic) energy to mechanically expand LLPs and to create new, very large TPs.

Three ranges (orders of magnitude) for the pore lifetime are found:

(1) 10 to 100 ns
a. Clean molecular dynamics (MD) models
b. Made only from mathematics and observed in silico
c. No evidence of metastability; MD “clean”, no “dirt”
   (2) Milliseconds to seconds
a. Pure artificial lipid bilayer membranes (BLM)
b. May contain contaminants
c. Melnikov experiments worried about contaminants

(3) Seconds to minutes
a. Real cells with real membranes
b. Cell interior is “overcrowded” with molecules
c. Lots of macromolecules hitting against membrane

The “two pore” model is supported by:

(1) More experimental evidence for two pore types

(2) Wide range of cell membrane recovery times

(3) Consistent MD recovery times of ˜10-100 ns

(4) Correlation of recovery with “contaminants”

(5) Concept/physics of macromolecule insertion

In summary TP to LLP to TP transitions:

1. TPs created by EP (here, single electric pulse)

2. Many insertion attempts, success rare
a. LLPs small fraction of TPs during pulse (implication of modeling and experiments)

3. LLPs can expand electrically or mechanically
a. Molecule segment escape by tension increase
b. LLPs transition to TPs, but tension then large

4. Transition back to TPs, held open by tension

The mechanistic hypothesis has metastable TPs and LLPs with 9 orders of magnitude lifetime differences (100 ns vs. 100 s).

The three phases of poration can be summarized as follows:

1. Phase 0: Pre-pulse, spontaneous TPs, on and off

2. Phase 1: EP pulse creates many TPs electrically

3. Phase 2: Post-pulse, ˜100 LLPs emerge, persist

4. Phase 3: LLPs transport small ions into/out of cell
a. Cell osmotic pressure difference slowly changes
b. Later pressure changes abruptly accelerates
c. Increased membrane tension:
   Expands ˜100 LLPs
   Creates many new TPs

### Example 2

### Cells can be Electroporated Using a Single Electrical Pulse

### Methods

### 1. Cell Treatments in Microfluidic Chambers

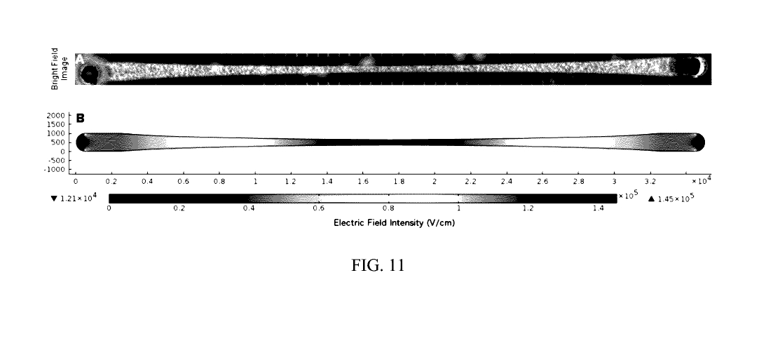

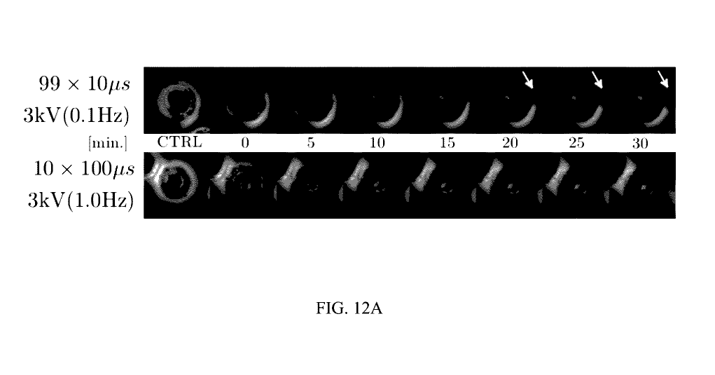

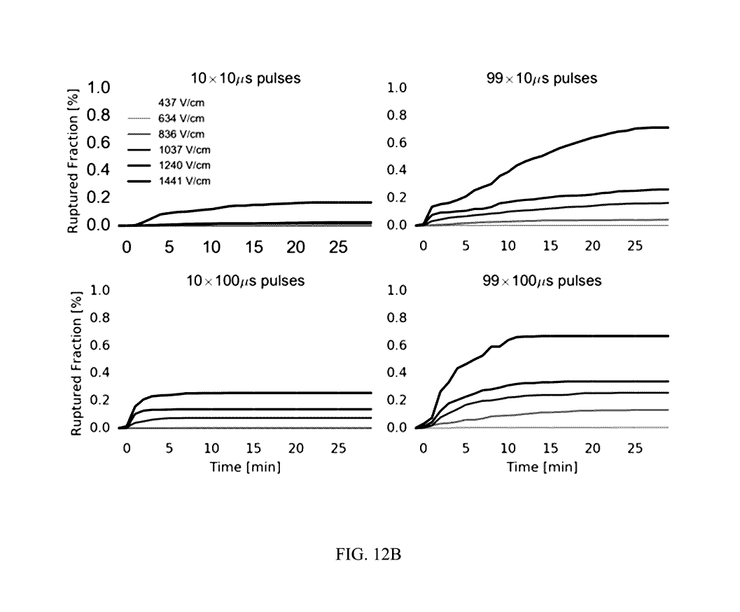

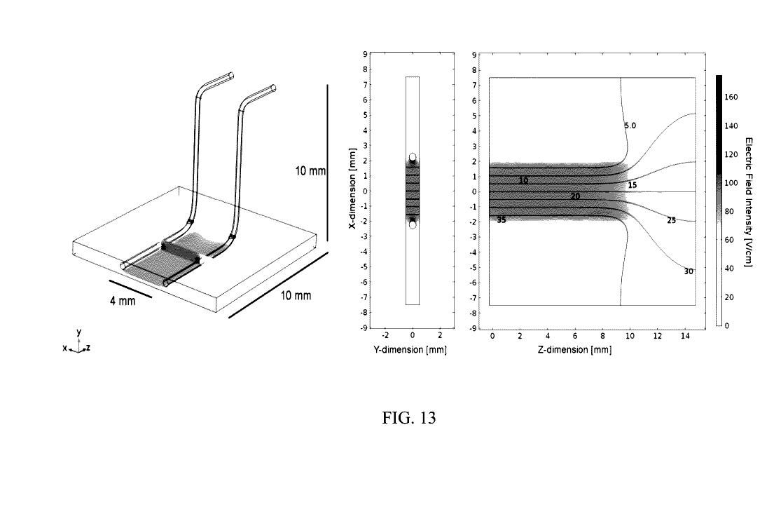

Data were obtained from two experimental setups: in a microfluidic device and in a growth chamber. Within the microfluidic device, Chinese hamster ovarian (CHO) cells were seeded at a density between 2-5×10⁶ cells/mL inside a microfluidic chip and allowed to adhere overnight. The channel height is approximately 90 μm and tapered along its length (approximately 3-4 cm) to generate a continuous electric field gradient across the length of the channel (FIG. 11). It was observed that a cell leakage event (FIG. 12) occurred over time and that it always preceded a large fluorescent intensification when it occurred. When quantified for each treatment, these leakage events occurred with increasing frequency as the electric field intensity was increased, above a certain threshold (FIG. 12). The observed leakage events occur differently, even under similar treatment times, using different pulse width pulses. It was observed that 99×10 μs pulses would elicit a larger fraction of cells exhibiting leakage than 10×100 μs pulses (FIG. 12), indicating that the leakage event is dependent on both the pulse width and amplitude of the applied electric field.

### 2. Cell Treatments in Open-Well Chambers

The growth-chamber used in the second experimental setup is based on a Lab-Tek II chamber (FIG. 11) into which two platinum-iridium (90:10) wire electrodes are inserted to make electrical contact with the cell medium while mitigating electrochemical effects typically associated with metal electrodes in aqueous media (Loomis-Husselbee et al., Biochem. J., 277 (3): 883-885, 1991). To detect electroporation, propidium iodide (PI) was mixed with phosphate buffered saline (PBS) and this mixture was used as the buffer in which the cells were exposed to the electric field treatments.

### 3. Calibration of Fluorescence

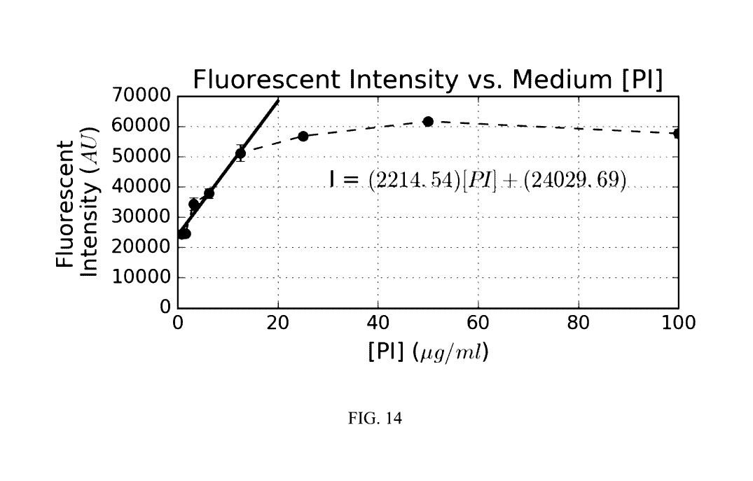

The fluorescence intensity observed during each treatment may be correlated with a concentration of PI. To obtain this calibration curve, rat hepatocellular carcinoma cells (H4IIE) were seeded at 7×10⁴ cells/ml in Lab-Tek II chambers and allowed to settle and adhere for 2-4 hours at 37° C. and 5% CO₂ to allow sufficient time for them to adhere to the chamber base while remaining largely spherical. Following incubation, the medium was removed from the chambers and, while on the microscope stage, a 0.1% Triton solution in phosphate buffered saline (PBS) with various concentrations of PI was added to the chamber while an imaging sequence was performed simultaneously. The Triton solution chemically permeabilized the cell membrane and was used as a positive control to generate the calibration curve (FIG. 14).

### 4. Single-Pulse Treatments

Electroporation was found to be effectively performed using single-pulse schemes to electroporate cells. Using a single pulse rather than the conventional pulse trains enables electroporation to be performed using significantly less energy than that which is currently used. However, a delayed response may present several minutes following treatment due to the initial destabilization of the membrane allowing molecular transport to occur that will over time destabilize the whole cell. In the treatments performed in vitro, this transport was visualized as a leakage of fluorescent cytosolic components entering the extracellular space (FIG. 14). We have shown that we are able to exploit this phenomenon using a single electrical pulse in vitro to sufficiently destabilize the cell membrane to a degree where is it not able to recover and ultimately destabilizes the entire cell following treatment with a single electrical pulse.

### 5. Monophasic and Biphasic Fluorescence Intensification

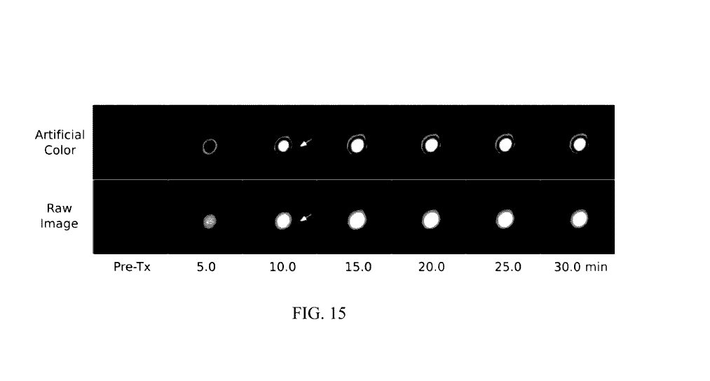

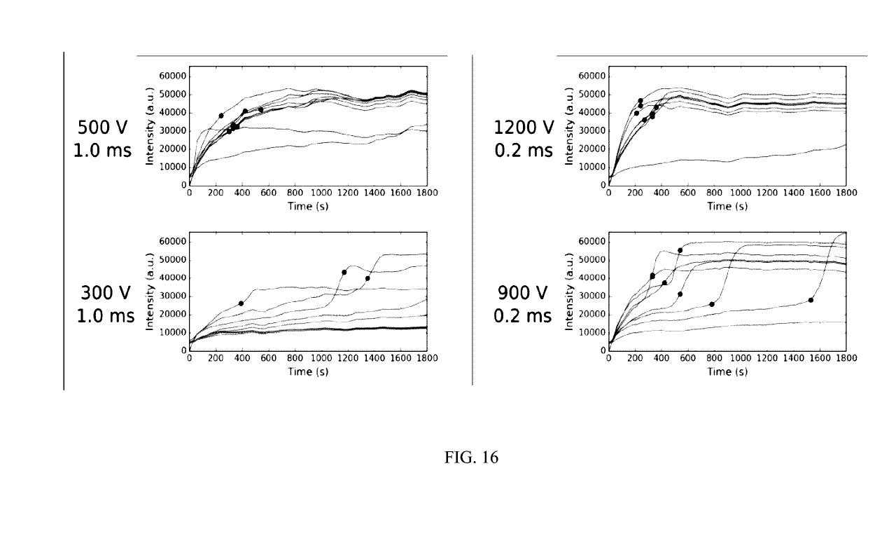

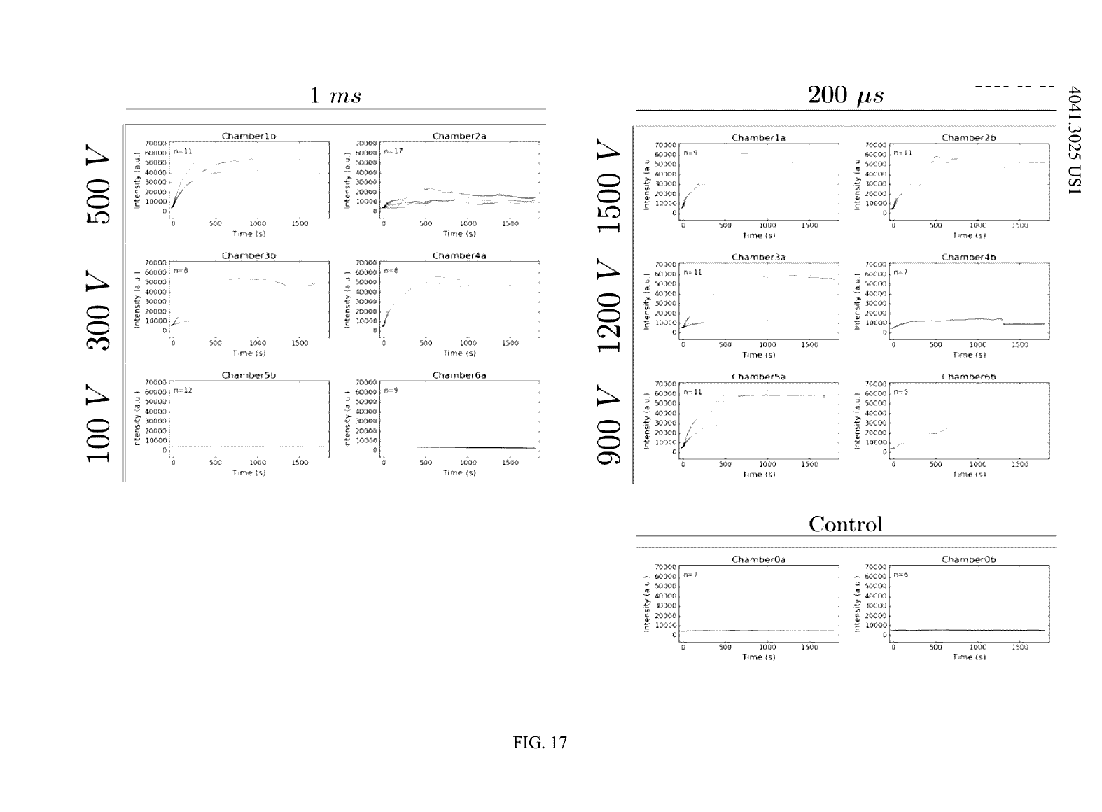

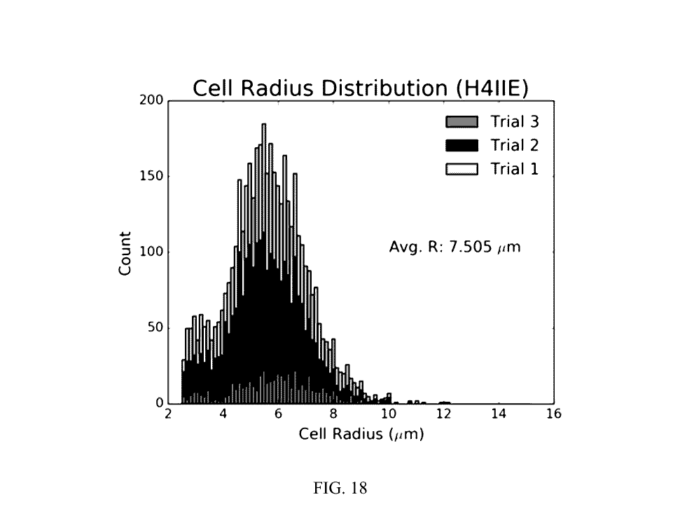

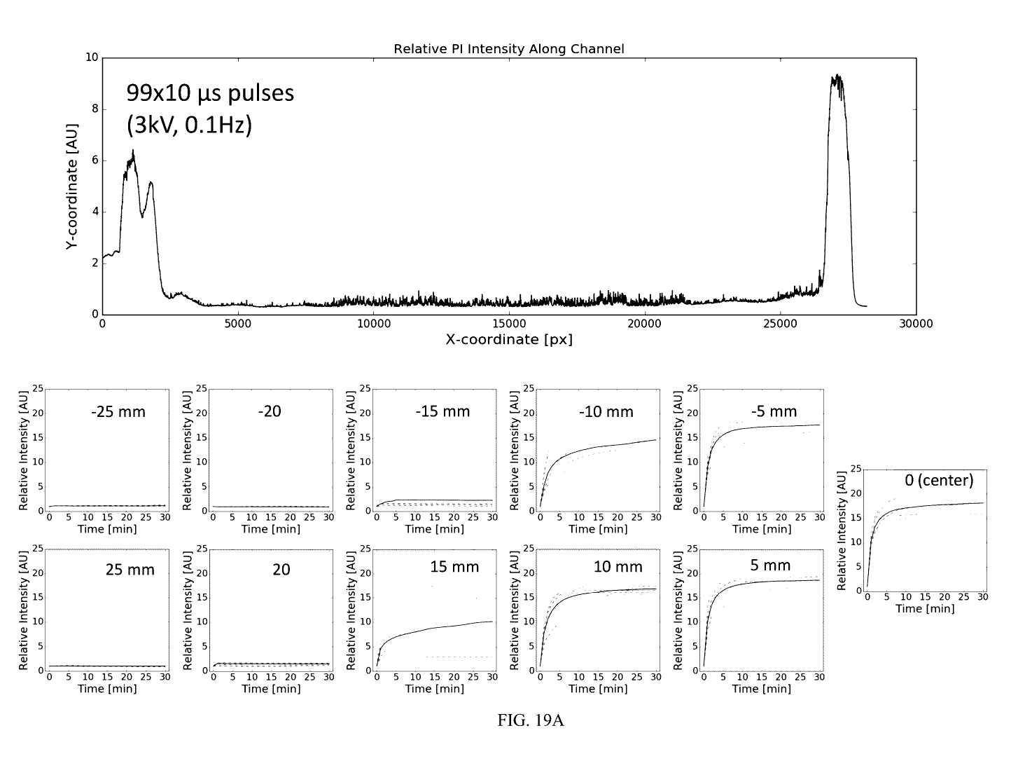

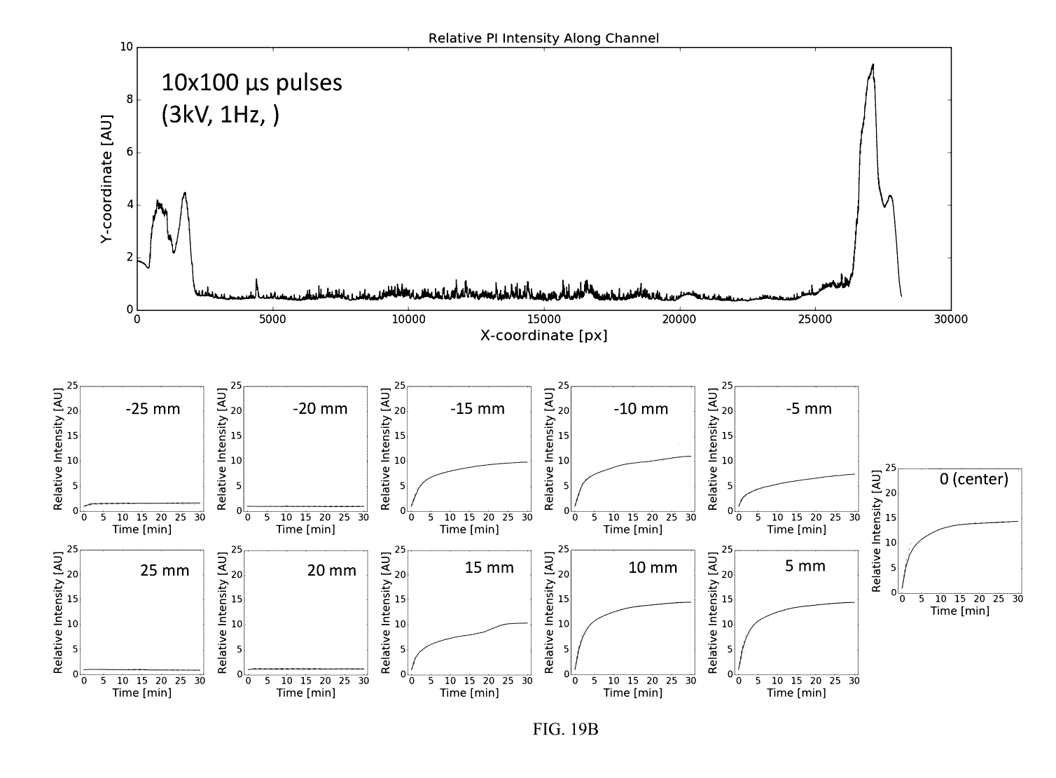

Further analysis of the individual cells exposed to different duration and amplitude electrical pulses revealed that the fluorescence intensification observed in vitro may occur either in a monophasic or biphasic manner that depends on the electrical pulse duration and amplitude selected to apply the electrical pulse. For a given pulse duration, the PI uptake and subsequent fluorescence may appear as a continuously increasing function that will reach an asymptote value over time. However, for a smaller range of electric field intensities, the initial pulse will result in an initial small fluorescence intensification of the cell, alluding to a small amount of PI entering the cell. However, a second inflection point in the fluorescence profile occurs several minutes post-treatment at which point the cell attains the fluorescent intensity of cell exposed to a pulse that would cause a monophasic intensification. For example, FIG. 16 shows intensification profiles for cells exposed to various electric field treatments. For the cells exposed to pulses of 500 V (1.1 to 1.25 kV/cm) for 1.0 ms and 1200 V (2.64 to 3 kV/cm) for 0.2 ms, a monophasic increase in fluorescence intensity occurs, reaching an asymptote after approximately 10 min. However, by maintaining the pulse duration but lowering the applied voltage from 500 V (1.1 to 1.25 kV/cm) to 300 V (0.66 to 0.75 kV/cm) for the 1.0 ms pulse and from 1200 V (2.64 to 3 kV/cm) to 900 V (1.98 to 2.25 kV/cm) for the 0.2 ms pulse, the cells still became electroporated to similar degrees as those exposed to the higher amplitude pulses, yet with significantly lower dissipated energy. However, these smaller pulses generate biphasic responses in the PI uptake as visualized by the fluorescence intensification over time. Additionally, cell leakage occurred in cells exposed to each of the single pulse treatments in FIG. 16. The image frame in the imaging sequence in which this leakage was first detectable, as demonstrated by FIG. 15, is marked in FIG. 16 as a black circle on each of the profiles exhibiting this behavior. For each biphasic pulse, this leakage event was detected just before or during the second inflection point in the fluorescence intensification profiles and is always preceded by cellular swelling. These two observations together suggest that the initial exposure to the electrical pulse destabilizes the cell membrane yet does not entirely render it permeable to PI to the degree a larger-amplitude pulse would. A long-lived pore (LLP) mechanism would explain these observations by describing the initial cell permeabilization through the stochastic generation of pores of a range of radii. Most of these pores quickly reseal, though some may remain open, as indicated by the increasing fluorescence intensity profiles prior to the second inflection points (FIG. 16). This population of stable pores allows for the exchange of water molecules and ions along osmotic gradients between the intracellular space and the extracellular medium. The water and molecules moving into the cell expand the cell and, when the pressure inside the cell overcomes the mechanical strain exerted by the cell membrane, the membrane ruptures and the cytoplasmic components, containing PI bound to double-stranded nucleic acids, leaks through the rupture into the extracellular space, indicated in FIG. 15.

### CONCLUSION

We have herein shown that cells may be effectively electroporated using a single electrical pulse. We have demonstrated that a lower-than-conventional electroporation regime exists where cell permeabilization monitored using PI fluorescence has a biphasic response that correlates to an initial electroporation event followed by swelling and leakage events that render the target cells as permeable as higher amplitude pulses. This work represents a new regime of pulse parameters for application that are able to decrease the amount of thermal damage to the target cells by dramatically decreasing the total energy applied during an electroporation-based treatment.

The relevance of this work to medicine includes: using post-electroporation swelling as a treatment that minimizes muscle contractions due to a single pulse being applied in clinical electroporation-based treatments and therapies and allowing the non-thermally treated tissue region to be increased beyond what present treatments allowing because thermal damage is minimized. The relevance of this work also extends to combining single-pulse electroporation schemes with adjuvants to further enhance membrane permeability, minimizing tissue necrosis because thermal damage is minimized and potentially enhancing the ratio of apoptotic cell death to necrotic cell death with the treated tissue region which is associated with certain clinical advantages.

### REFERENCES

[1] D. A. Stewart, T. R. Gowrishankar, and J. C. Weaver. Transport lattice approach to describing cell electroporation: use of a local asymptotic model. IEEE Transactions on Plasma Science, 32:1696-1708, 2004.
[2] T. R. Gowrishankar, A. T. Esser, Z. Vasilkoski, K. C. Smith, and J. C. Weaver. Microdosimetry for conventional and supra-electroporation in cells with organelles. Biochem. Biophys. Res. Commun., 341:1266-1276, 2006.
[3] K. C. Smith and J. C. Weaver. Active mechanisms are needed to describe cell responses to submicrosecond, megavolt-per-meter pulses: Cell models for ultrashort pulses. Biophys. J., 95:1547-1563, 2008.
[4] A. T. Esser, K. C. Smith, T. R. Gowrishankar, Z. Vasilkoski, and J. C. Weaver. Mechanisms for the intracellular manipulation of organelles by conventional electroporation. Biophys. J., 98:2506-2514, 2010.
[5] J. C. Weaver, K. C. Smith, A. T. Esser, R. S. Son, and T. R. Gowrishankar. A brief overview of electroporation pulse strength—duration space: A region where additional intracellular effects are expected. Bioelectrochemistry, 87:236-243, 2012.
[6] J. C. Weaver. Electroporation: A general phenomenon for manipulating cells and tissue. J. Cellular Biochem., 51:426-435, 1993.
[7] J. C. Weaver. Electroporation theory—concepts and mechanisms. In J. A. Nickloff, editor, Electroporation Protocols for Microorganisms, pages 1-26. Humana Press, Totowa, N.J., 1995.
[8] M.-P. Rols and J. Teissié. Electropermeabilization of mammalian cells to macromolecules: control by pulse duration. Biophys. J., 75:1415-1423, 1998.
[9] T. R. Gowrishankar, U. Pliquett, and R. C. Lee. Dynamics of membrane sealing in transient electropermeabilization of skeletal muscle membranes. Ann. N.Y. Acad. Sci., 888:195-210, 1999.
[10] M. Pavlin, V. Leben, and D. Miklavcic. Electroporation in dense cell suspension: Theoretical and experimental analysis of ion diffusion and cell permeabilization. Biochim. Biophys. Acta, 1770:12-23, 2007.
[11] A. G. Pakhomov, E. Gianulis, P. T. Vernier, I. Semenov, S. Xiao, and O. Pakhomova. Multiple nanosecond electric pulses increase the number but not the size of long-lived nanopores in the cell membrane. Biochim. Biophys. Acta, 1848:958-966, 2015.
[12] L. H. Wegner, W. Frey, and A. Silve. Electroporation of DC-3F cells is a dual process. Biophysical J., 108:1660-1671, 2015.
[13] Y. Demiryurek, M. Nickaeen, M. Zheng, M. Yu, J. D. Zahn, D. I. Shreiber, H. Lin, and J. W. Shan. Transport, resealing, and re-poration dynamics of two-pulse electroporation-mediated molecular delivery (accepted). Biochim. Biophys. Acta, 2015.
[14] P. T. Vernier, Y. Sun, and M. A. Gundersen. Nanoelectropulse-driven membrane perturbation and small molecule permeabilization. BMC Cell Biol., 7:37-1-37-16, 2006.
[15] A. G. Pakhomov, R. Shevin, J. A. White, J. F. Kolb, O. N. Pakhomova, R. P. Joshi, and K. H. Schoenbach. Membrane permeabilzation and cell damage by ultrashort electric field shocks. Arch. Biochem. Biophys., 465:109-118, 2007.
[16] A. G. Pakhomov, J. F. Kolb, J. A. White, R. P. Joshi, S. Ziao, and K. H. Schoenbach. Long-lasting membrane permeabilzation in mammalian cells by nanosecond pulsed electric field (nsPEF). Bioelectromagnetics., 28:655-663, 2007.
[17] A. G. Pakhomov, A. M. Bowman, B. L. Ibey, F. M. Andre, 0. N. Pakhomova, and K. H. Schoenbach. Lipid nanopores can form a stable, ion channel-like conduction pathway in cell membrane. Biochem. Biophys. Res. Commun., 385:181-186, 2009.
[18] T. R. Gowrishankar and J. C. Weaver. An approach to electrical modeling of single and multiple cells. Proc. Nat. Acad. Sci., 100:3203-3208, 2003.
[19] K. C. Smith. A unified model of electroporation and molecular transport. Massachusetts Institute of Technology, http://dspace.mit.edu/bitstream/handle/1721.1/63085/725958797.pdf.
[20] T. R. Gowrishankar, K. C. Smith, and J. C. Weaver. Transport-based biophysical system models of cells for quantitatively describing responses to electric fields. Proc IEEE, 101:505-517, 2013.
[21] K. C. Smith and J. C. Weaver. Electrodiffusion of molecules in aqueous media: A robust, discretized description for electroporation and other transport phenomena. IEEE Trans. Biomed. Engr., 59:1514-1522, 2012.
[22] K. C. Smith, R. S. Son, T. R. Gowrishankar, and J. C. Weaver. Emergence of a large pore subpopulation during electroporating pulses. Bioelectrochemistry, 100:3-10, 2014.
[23] R. S. Son, K. C. Smith, T. R. Gowrishankar, P. T. Vernier, and J. C. Weaver. Basic features of a cell electroporation model: Illustrative behavior for two very different pulses. J. Membrane Biol., 247:1209-1228, 2014.
[24] J. C. Weaver, K. C. Smith, R. S. Son, and T. R. Gowrishankar. Continuum modeling for bioelectrics (submitted). In H. Akiyama & R. Hellerr, editor, Bioelectrics. Springer, Heidelgerg, 2015.
[25] E. A. Evans. New membrane concept applied to the analysis of fluid shear—and micropipette-deformed red blood cells. Biophysical J., 13:941-954, 1973.
[26] E. Evans, V. Heinrich, F. Ludwig, and W. Rawicz. Dynamic tension spectroscopy and strength of biomembranes. Biophys. J., 85:2342-2350, 2003.
[27] E. Evans and B. A Smith. Kinetics of hole nucleation in biomembrane rupture. New J. Phys., 13:095010, 2011.
[28] D. P. Tieleman, H. Leontiadou, A. E. Mark, and S.-J. Marrink. Simulation of pore formation in lipid bilayers by mechanical stress and electric fields. J. Am. Chem. Soc., 125:6382-6383, 2003.
[29] D. P. Tieleman. The molecular basis of electroporation. BMC Biochemistry, 5:10, 2004.
[30] P. T. Vernier, M. J. Ziegler, Y. Sun, W. V. Chang, M. A. Gundersen, and D. P. Tieleman. Nanopore formation and phosphatidylserine externalization in a phospholipid bilayer at high transmembrane potential. J. Am. Chem. Soc., 128:6288-6289, 2006.
[31] P. T. Vernier and M. J. Ziegler. Nanosecond field alignment of head group and water dipoles in electroporating phospholipid bilayers. J. Phys. Chem. B, 111:12993-12996, 2007.
[32] M. J. Ziegler and P. T. Vernier. Interface water dynamics and porating fields for phospholipid bilayers. J. Phys. Chem. B, 112:13588-13596, 2008.
[33] Z. A. Levine and P. T. Vernier. Life cycle of an electropore: Field-dependent and field-independent steps in pore creation and annihilation. J. Memb. Biol., 236:27-36, 2010.
[34] M. L. Fernandez, M. Risk, R. Reigada, and P. T. Vernier. Size-controlled nanopores in lipid membranes with stabilizing electric fields. Biochem. Biophys. Res. Commun., 423:325-330, 2012.
[35] M.-C. Ho, M. Casciola, Z. A. Levine, and P. T. Vernier. Molecular dynamics simulations of ion conductance in field-stabilized nanoscale lipid electropores. J. Phys. Chem. B, 117:11633-11640, 2013.
[36] J. Newman. Resistance for flow of current to a disk. J. Electrochem. Soc., 113:501-502, 1966.
[37] J. E. Hall. Access resistance of a small circular pore. J. Gen. Physiol., 66:531-532, 1975.
[38] J. C. Weaver and Y. A. Chizmadzhev. Theory of electroporation: A review. Bioelectrochem. Bioenerget., 41:135-160, 1996.
[39] C. Dekker. Solid-state nanopores. Nature Nanotech., 2:209-215, 2007.
[40] M. Wanunu, W. Morrison, Y. Rabin, A. Y. Grosberg, and A. Meller. Electrostatic focusing of unlabelled DNA into nanoscale pores using a salt gradient. Nature Biotechnology, 5:160-165, 2010.
[41] M. Firnkes, D. Pedone, J. Knezevic, M. Döblinger, and U. Rant. Electrically facilitated translocations of proteins through silicon nitride nanopores: Conjoint and competitive action of diffusion, electrophoresis, and electroosmosis. Nano Lett., 10:2162-2167, 2010.
[42] K. Kinosita, I. Ashikawa, N. Saita, H. Yoshimura, H. Itoh, K. Nagayma, and A. Ikegami. Electroporation of cell membrane visualized under a pulsed-laser fluorescence microscope. Biophys. J., 53:1015-1019, 1988.
[43] W. Frey, J. A. White, R. O. Price, P. F. Blackmore, R. P. Joshi, R. Nuccitelli, S. J. Beebe, K. H. Schoenbach, and J. F. Kolb. Plasma membrane voltage changes during nanosecond pulsed electric field exposures. Biophys. J., 90:3608-3615, 2006.
[44] B. Flickinger, T. Berghofer, P. Hohenberger, C. Eing, and W. Frey. Transmembrane potential measurements on plant cells using the voltage-sensitive dye ANNINE-6. Protoplasma, 247:3-12, 2010.
[45] D. Mycue, J. Zahn, M. Zahn, and J. C. Weaver. Interaction mechanism of electric fields with cells: Alteration of binding site access of membrane proteins. In M. Blank, editor, Electricity and Magnetism in Biology and Medicine, pages 773-776. San Francisco Press, San Francisco, 1993.
[46] V. A. Parsegian. Energy of an ion crossing a low dielectric membrane: Solutions to four relevant electrostatic problems. Nature, 221:844-846, 1969.
[47] V. A. Parsegian. Ion-membrane interactions as structural forces. Ann. N. Y. Acad. Sci., 264:161-174, 1975.
[48] J. C. Weaver, R. Vanbever, T. E. Vaughan, and M. R. Prausnitz. Heparin alters transdermal transport associated with electroporation. Biochem. Biophy. Res. Comm., 234:637-640, 1997.
[49] R. Vanbever, M. R. Prausnitz, and V. Preat. Macromolecules as novel transdermal transport enhancers for skin electroporation. Pharm. Res., 14:638-644, 1997.
[50] S. R. McGuffee and A. H. Elcock. Diffusion, crowding and protein stability in a dynamic molecular model of the bacterial cytoplasm. PLoS Comp. Biol., 6:e1000694, 2010.
[51] Y. Mouneimne, P-F. Tosi, Y. Gazitt, and C. Nicolau. Electro-insertion of xeno-glycophorin into the red blood cell membrane. Biochem. Biophys. Res. Comm., 159:34-40, 1989.
[52] M. Zeira, P-F. Tosi, Y. Mouneimne, J. Lazarte, L. Sneed, D. J. Volsky, and C. Nicolau. Full-length CD4 electro-inserted in the erythrocyte membrane as a long-lived inhibitor of infection by human immunodeficiency virus. Proc. Nat. Acad. Sci., 88:4409-4413, 1991.
[53] C. Nicolau, Y. Mouneimne, and P.-F. Tosi. Electroinsertion of proteins in the plasma membrane of red blood cells. Analytic. Biochem., 1993:1-10, 1993.
[54] K. E. Ouagari, J. Teissie, and H. Benoist. Glycophorin a protects K562 cells from natural killer cell attack. J. Biol. Chem., 270:26970-26975, 1995.
[55] S. M. Kennedy, Z. Ji, J. C. Hedstrom, J. H. Booske, and S. C. Hagness. Quantitation of electroporation uptake kinetics and electric field heterogeneity effects in cells. Biophys. J., 94:5018-5027, 2008.
[56] A. Diz-Munoz, D. A. Fletcher, and O. D. Weiner. Use the force: membrane tension as an organizer of cell shape and motility. Trends Cell Biol., 23:47-53, 2013.
[1A] R. V. Davalos, L. M. Mir, and B. Rubinsky. Tissue ablation and irreversible electroporation. Ann. Biomed. Eng., 33:223-231, 2005.
[2B] C. Jiang, R. V. Davalos, and J. C. Bischof. A review of basic to clinical studies of irreversible electro-poration therapy. Trans. Biomed. Eng., 62:4-20, 2015.
[3B] L. Galluzzi, I. Vitale, J. M. Abrams, E. S. Alnemri, E. H. Baehrecke, M. V. Blagosklonny, T. M. Daw-son, V. L. Dawson, W. S. El-Deiry, S. Fulda, E. Gottlieb, D. R. Green, M. O. Hengartner, O. Kepp, R. A. Knight, S. Kumar, S. A. Lipton, X. Lu, F. Madeo, W. Malorni, P. Mehlen, G. Nunez, M. E. Peter, M. Piacentini, D. C. Rubinsztein, Y. Shi, H-U Simon, P. Vandenabeele, E. White, J. Yuan, B. Zhivo-tovsky, G. Melino, and G. Kroemer. Molecular definitions of cell death subroutines: Recommendations of the nomenclature committee on cell death 2012. Cell Death Diff., 19:107-120, 2012.
[4B] W. Ren, N. M. Sain, and S. J. Beebe. Nanosecond pulsed electric fields (nsPEFs) activate intrinsic caspase-dependent and caspase-independent cell death in Jurkat cells. Biochem. Biophys. Res. Com-mun., 421:808-812, 2012.
[5B] M. Faroja, M. Ahmed, L. Appelbaum, E. Ben-David, M. Moussa, J. Sosna, I. Nissenbaum, and S. N. Goldberg. Irreversible electroporation ablation: is all the damage nonthermal? Radiology, 266:462-470, 2013.
[6B] M. J. C. van Gemert, P. G. K. Wagstaff, D. M. de Bruin, T. G. van Leeuwen, A. C. van der Wal, M. Heger, and C. W. M. van der Geld. Irreversible electroporation: just another form of thermal therapy? The Prostate, 75:332-335, 2015.
[7B] F. C. Henriques. Studies of thermal injury. V. the predictability and the significance of thermally induced rate processes leading to irreversible epidermal injury. Arch. Pathol., 43:489-502, 1947.
[8B] A. Golberg, B. G. Bruinsma, B. E. Uygunl, and M. L. Yarmush. Tissue heterogeneity in structure and conductivity contribute to cell survival during irreversible electroporation ablation by electric field sinks. Sci. Reports, 5:8485-1-8485-7, 2015.
[9B] J. J. Breedlove. Heat transfer between blood vessles and perfused tissue during hyperthermia therapy. Master's thesis, MIT, Cambridge, Mass., 1997. Dept. of Mech. Engr.
[10B] J. Gehl, T. Skovsgaard, and L. M. Mir. Vascular reactions to in vivo electroporation: characterization and consequences for drug and gene delivery. Biochim. Biophys. Acta, 1428:233-240, 1999.
[1C] I. G. Abidor, V. B. Arakelyan, L. V. Chernomordik, Y. A. Chizmadzhev, V. F. Pastushenko, and M. R. Tarasevich, “246-Electric breakdown of bilayer lipid membranes I. The main experimental facts and their qualitative discussion,” Bioelectrochemistry Bioenerg, vol. 6, no. 1, pp. 37-52, 1979.
[2C] A. M. Lebar, N. A. Kopitar, K. Ihan, G. Sersa, and D. Miklavcic, “Significance of treatment energy in cell electropermeabilization,” Electro- and Magnetobiology, vol. 17, no. 2, pp. 255-262, 1998.
[3C] H. Shafiee, P. a Garcia, and R. V Davalos, “A preliminary study to delineate irreversible electroporation from thermal damage using the arrhenius equation.,” J Biomech Eng, vol. 131, no. 7, p. 074509, July 2009.
[4C] E. Neumann, M. Schaefer-Ridder, Y. Wang, and P. H. Hofschneider, “Gene transfer into mouse lyoma cells by electroporation in high electric fields.,” EMBO J, vol. 1, no. 7, pp. 841-845, 1982.
[5C] R. V Davalos, L. M. Mir, and B. Rubinsky, “Tissue Ablation with Irreversible Electroporation,” Ann Biomed Eng, vol. 33, no. 2, pp. 223-231, February 2005.
[6C] L. M. Mir, L. F. Glass, G. Sersa, J. Teissie, C. Domenge, D. Miklavcic, M. J. Jaroszeski, S. Orlowski, D. S. Reintgen, Z. Rudolf, M. Belehradek, R. Gilbert, M. P. Rols, J. Belehradek, J. M. Bachaud, R. DeConti, B. Stabuc, M. Cemazar, P. Coninx, and R. Heller, “Effective treatment of cutaneous and subcutaneous malignant tumours by electrochemotherapy.,” Br J Cancer, vol. 77, no. 12, pp. 2336-42, 1998.
[7C] J. F. Edd and R. V Davalos, “Mathematical modeling of irreversible electroporation for treatment planning.,” Technol Cancer Res Treat, vol. 6, no. 4, pp. 275-86, August 2007.
[8C] R. C. G. Martin, D. Kwon, S. Chalikonda, M. Sellers, E. Kotz, C. Scoggins, K. M. McMasters, and K. Watkins, “Treatment of 200 Locally Advanced (Stage III) Pancreatic Adenocarcinoma Patients With Irreversible Electroporation,” Ann Surg, vol. 262, no. 3, pp. 486-494, 2015.
[9C] Jourbachi et al., “Irreversible Electroporation (Nanoknife) in Cancer Treatment,” Gastrointest. Interv. 3:8-18, 2014.
[10C] Jiang et al., “A Reviewing of Basic to Clinical Studies of Irreversible Electroporation Therapy,” IEEE Transactions on Biomedical Engineering, 62(1): 4-20, 2015.
[11C] Wendler et al., “Irreversible Electroporation (IRE): Standardization of Terminology and Reporting Criteria for Analysis and Comparison,” Polish Journal of Radiology 81: 54-64, 2016.
[12C] J. W. Loomis-Husselbee, P. J. Cullen, R. F. Irvine, and A. P. Dawson, “Electroporation can cause artefacts due to solubilization of cations from the electrode plates. Aluminum ions enhance conversion of inositol 1,3,4,5-tetrakisphosphate into inositol 1,4,5-trisphosphate in electroporated L1210 cells.,” Biochem J, vol. 277 (Pt 3, pp. 883-885, 1991.
[13C] Son et al. “Modeling a Conventional Electroporation Pulse Train: Decreased a Pore Number, Cumulative Calcium Transport and Example of Electrosensitization,” IEEE Transaction on Biomedical Engineering 63(3): 571-580, 2016.

While this invention has been particularly shown and described with references to preferred embodiments thereof, it will be understood by those skilled in the art that various changes in form and details may be made therein without departing from the scope of the invention encompassed by the appended claims.

1. A method of ablating a target tissue in a subject in need thereof, wherein the method comprises:
a) placing one or more electrodes within or near the target tissue; and
b) applying a single electrical pulse to the target tissue in an amount which is sufficient to induce cell permeabilization and cell death, wherein the primary mechanism of cell death is electroporation.

2. A method of ablating a target tissue in a subject in need thereof, comprising the steps of:
a) placing one or more electrodes within or near the target tissue; and
b) applying a single electrical pulse to the target tissue in an amount which is sufficient to induce biphasic cell permeabilization of the cells of the target tissue, wherein cell death is induced, and wherein the biphasic cell permeabilization comprises electroporation and post-electroporation osmotic swelling and leakage of the cells.

3. The method of claim 1, wherein the amplitude and/or duration of the pulse is less than that of an IRE pulse protocol that induces monophasic cell permeabilization for the same target tissue.

4. The method of claim 1, comprising placing a first electrode and a second electrode such that the target tissue is positioned between the first and second electrodes.

5. The method of claim 1, wherein the one or more electrodes are part of a single device.

6. (canceled)

7. The method of claim 1, wherein the single electrical pulse results in less thermal damage than that induced by an IRE pulse protocol that induces monophasic cell permeabilzation for the same target tissue.

8. The method of claim 1, wherein the single electrical pulse is applied in an amount which maintains the temperature of the target tissue at about 65° C. or less.

9. (canceled)

10. (canceled)

11. The method of claim 1, wherein the duration of the pulse is between about 1 microsecond and about 50 milliseconds.

12-14. (canceled)

15. The method of claim 1, wherein the electric field strength is between about 100 to about 5000 V/cm.

16-18. (canceled)

19. The method of claim 1, wherein the target tissue is malignant tissue.

20. The method of claim 1, wherein the target tissue is non-malignant tissue.

21. The method of claim 1, wherein the target tissue is a tumor.

22. (canceled)

23. (canceled)

24. The method of claim 21, wherein the tumor is a soft tissue tumor.

25. The method of claim 21, wherein the tumor is selected from the group consisting of a lung, liver, kidney, pancreatic, prostate, breast, colorectal, peri-biliary, melanoma, head and neck, and thyroid tumors.

26. (canceled)

27. The method of claim 1, wherein subject is suffering from breast cancer, colorectal liver metastasis, head and neck cancer, hepatocellular carcinoma, pancreatic cancer, bone cancer, lung cancer, soft tissue cancer, melanoma, peri-biliary tumor, prostate cancer, renal cell carcinoma, renal mass or uveal melanoma.

28. (canceled)

29. (canceled)

30. The method of claim 25, wherein the volume of the target tumor is about 10 cm³ or greater.

31. The method of claim 30, wherein the volume of the target tumor is about 30 cm³ or greater.

32. The method of claim 1, wherein muscular contractions in the subject are reduced as compared to those that occur using an IRE pulse protocol that induces monophasic cell permeabilzation for the same target tissue.

33. The method of claim 32, wherein a neuromuscular blocking agent is not administered to the subject.

34. The method of claim 1, wherein an adjuvant is administered to the subject before, during or after the application of the electrical pulse.

35-38. (canceled)

39. A method of ablating a target tissue in a subject in need thereof, comprising the steps of:
a) placing one or more electrodes within or near the target tissue; and
b) applying a plurality of electrical pulses to the target tissue in an amount which is sufficient to induce biphasic cell permeabilization of the cells of the target tissue, wherein cell death is induced and wherein the biphasic cell permeabilization comprises electroporation and post-electroporation osmotic swelling and leakage of the cells,
wherein the plurality of electrical pulses are each applied at least about 0.1 microsecond to at least about one minute apart,
and further wherein the plurality of electrical pulses is less than eight pulses.

40. The method of claim 39, wherein the plurality of electrical pulses is five pulses or less.

41. (canceled)

42. (canceled)

43. The method of claim 39, wherein the plurality of electrical pulses is two pulses.

44. The method of claim 39, wherein the amplitude and/or duration of each pulse is less than that of an IRE pulse protocol that induces monophasic cell permeabilization for the same target tissue.

45-47. (canceled)

48. The method of claim 39, wherein the method results in less thermal damage than that induced by an IRE pulse protocol that induces monophasic cell permeabilzation.

49. The method of claim 39, wherein the plurality of electrical pulses are applied in an amount which maintains the temperature of the target tissue at about 65° C. or less.

50. (canceled)

51. (canceled)

52. The method of claim 39, wherein the duration of each pulse is between about 1 microsecond and about 50 milliseconds.

53-55. (canceled)

56. The method of claim 39, wherein the electric field strength for each pulse is between about 100 and about 5000 V/cm.

57-59. (canceled)

60. The method of claim 39, wherein the target tissue is malignant tissue.

61. The method of claim 39, wherein the target tissue is non-malignant tissue.

62-66. (canceled)

67. A method of inducing a high permeability state in a cell membrane wherein said method comprises applying an electroporation pulse to a cell,
wherein at a time during or after the electroporation pulse is applied, a plurality of long lived pores (LLPs) are formed in the cell membrane and the presence of the LLPs causes a change in the cell osmotic pressure difference,
and further wherein after the change in the cell osmotic pressure difference, mechanoporation occurs wherein a plurality of the LLPs expand and/or a plurality of new pores are formed, thereby inducing a high permeability state in a region of the outer cell membrane.

68. The method of claim 67, wherein a single electroporation pulse is applied.

69. The method of claim 67, wherein cell death occurs after the induction of the high permeability state.

70. The method of claim 67, wherein the plurality of new pores include transient pores (TPs).

71-79. (canceled)
